(19) United States
(12) Patent Application Publication
(10) Pub. No.: US 2026/0308823 A1
(43) Pub. Date: Oct. 8, 2026

(54) MESH WITH REINFORCED EDGES AND METHOD OF MAKING THE SAME

(71) Applicant: ATEX TECHNOLOGIES, INC., PINEBLUFF, NC (US)

(72) Inventors: Brian McMurray, Aberdeen, NC (US); Laura Godwin Dailey, Southern Pines, NC (US); Stephanie Booz Norris, Rocky Point, NC (US)

(21) Appl. No.: 19/473,829

(22) PCT Filed: Apr. 9, 2024
(86) PCT No.: PCT/US24/23708

§ 371 Date: Oct. 9, 2025

Related U.S. Application Data
(60) Provisional application No. 63/458,330, filed on Apr. 10, 2023.

(51) Int. Cl. D04B 21/10; D04B 21/20

(52) U.S. Cl. CPC D04B 21/10; D04B 21/20; D10B 2509/00

(57) ABSTRACT

A warp knit mesh and method of making is provided herein. A warp knit mesh includes a first section, the first section comprising a plurality of warp knit yarns extending in side-by side relation. The plurality of warp knit yams define at least a first knit pattern and a second knit pattern. The second knit pattern extends adjacent the first knit pattern and forms a smooth edge without cut yarns. Different shapes and knit structures may be formed accordingly.

### CROSS-REFERENCE TO RELATED APPLICATION(S)

This application claims priority to U.S. Provisional Application No. 63/458,330, entitled “Mesh With Reinforced Edges and Method of Making the Same”, filed Apr. 10, 2023; the contents of which is incorporated by reference herein in its entirety.

### FIELD OF THE INVENTION

Embodiments of the present invention relate generally to mesh and, more particularly, to medical mesh with reinforced, smooth edges to prevent fraying of the mesh due to friction, unraveling, or cutting.

### BACKGROUND OF THE INVENTION

Medical meshes may be used in medical procedures both internally and externally, for example to support internal tissues, or to provide external support to the dermas. Medical mesh may be manufactured in swatches, and then may be cut down to size for the need of the procedure. The edges of the mesh may fray due to the cuts formed in the mesh, and/or due to friction created between the edges of the mesh and contact points (e.g., internal organs, sutures, securing mechanisms, other medical devices (including other meshes or portions of the mesh itself), etc.). In some cases, the propensity for fraying can increase depending on various mesh characteristics like an increased pore size and/or decreased stitch count.

### BRIEF SUMMARY OF THE INVENTION

Embodiments of the present invention are directed towards mesh configured to resist fraying, such as due to cutting of the mesh and/or friction between the mesh and other objects. The mesh may be knit with reinforced areas formed within the mesh during formation to thereby define discrete sections defined at least partially by the reinforced areas. The mesh may be separated into discrete sections by cutting the mesh adjacent the reinforced edges and/or removing one or more tacking yarns. In some such embodiments, reinforced edges that are smooth and without cut yarns are thereby provided. Alternatively, in some embodiments, one or more knibs or barbs (which may have cut portions) may be designed to extend past the reinforced edges, such as may be desired for certain scenarios. In some embodiment, the mesh may be formed with voids adjacent the reinforced areas.

The reinforced edges are designed to prevent fraying and unraveling of the knit mesh, such as due to friction and rubbing. In this regard, by creating the reinforced areas at specific warp knit yarns within the large mesh, each individual mesh portion may comprise reinforced edges. This prevents future fraying of the edges such as which might otherwise occur after formation and cutting, and it keeps the desired characteristics of the mesh (e.g., stiffness, shapability, tension, etc.). In this regard, as noted herein, mesh is often cut to form and this cutting results in unwanted fraying and reduced characteristics of the mesh (e.g., tension, shapability, and other characteristics are affected). Alternatively, in some cases, laser or other welding is used to cut and melt the newly formed edges, which can occur during manufacturing (e.g., pre-fabricated) and/or after manufacturing (such as at the end user site). This, however, creates a stiff and rough edge that is abrasive and undesirable for many surgical locations.

In some situations, objects may be positioned through a void (e.g., a slit) in the mesh. This may be for various reasons, including, for example, securing of the mesh, use with a medical device, installation or otherwise shaping of the mesh. Fraying and friction may occur when an object is positioned through the mesh. However, with various embodiments of the present invention, the object may be so used with the reinforced edges of the mesh and fraying, snagging, or other undesirable outcomes may be avoided.

Further, in some embodiments, the overall mesh may be designed such that even during cutting, no extra fragments may be created between the two mesh portions. For example, the reinforced edges and/or the opposing mesh portion (which may also have a reinforced edge) may form smooth edges even, in some embodiments, without removal of cut yarn. Notably, various knitting techniques are described herein that provide for such smooth and reinforced edges. Such knitting techniques can be utilized to form any desirable shapes, including, for example, strips between voids (e.g., attached at both ends, detached at one end, detached at both ends), bands leading into strips, angled complex shapes, circles, angled or tapered structures, among many other shapes.

As noted herein, various embodiments of the present invention allow for tension and flexibility with the mesh even if the mesh is prepared to form. The reinforced edges enable the mesh to be stretched in all directions, even along the reinforced edges, unlike a selvage edge (or similar feature). To explain, a selvage edge is generally utilized for manufacturability and then later cut off, or otherwise removed and discarded. In this regard, the selvage edge may not impart characteristics (e.g., stability, flexibility, etc.) on the mesh. In contrast, the reinforced edge is configured to exhibit different characteristics than the rest of the mesh such as to prevent the mesh from unwinding, and fraying due to friction.

In an example embodiment, a warp knit mesh is provided. The warp knit mesh comprising: a first section, the first section comprising: a plurality of warp knit yarns, the plurality of warp knit yarns defining at least a first knit pattern and a second knit pattern. At least a portion of the first knit pattern extends along at least two adjacent courses or wales, and at least a portion of the second knit pattern extends adjacent the first knit pattern along one of the at least two adjacent courses or wales in a parallel manner. The portion of the second knit pattern, as extended, has a higher density of loops or underlaps than the portion of the first knit pattern, as extended, such that the extended portion of the second knit pattern forms a first reinforced edge of the first section of mesh.

In some embodiments, the plurality of warp knit yarns transition from the first knit pattern to the second knit pattern between a first course and a second course.

In some embodiments, the plurality of warp knit yarns transition from the first knit pattern to the second knit pattern between a first wale and a second wale.

In some embodiments, the plurality of warp knit yarns comprise a plurality of first warp knit yarns and at least one second warp knit yarn, wherein the at least one second warp knit yarn is adjacent the plurality of first warp knit yarns, and wherein the plurality of first warp knit yarns comprise the first knit pattern, and the at least one second warp knit yarn comprises the second knit pattern.

In some embodiments, the plurality of warp knit yarns form at least a portion of a third knit pattern that extends past the second knit pattern relative to the first knit pattern, wherein the portion of the third knit pattern comprises nibs or barbs extending past the first reinforced edge.

In some embodiments, the warp knit mesh further comprises a second section, wherein the second section comprises a plurality of second warp knit yarns extending in side-by-side relation, the plurality of second warp knit yarns defining at least a portion of a third knit pattern and a portion of a fourth knit pattern, wherein the portion of the third knit pattern extends along at least two adjacent courses or wales, and wherein the portion of the fourth knit pattern extends adjacent the third knit pattern along one of the at least two adjacent courses or wales in a parallel manner, wherein the portion of the fourth knit pattern, as extended, has a higher density of loops or underlaps than the portion of the third knit pattern, as extended, such that the extended portion of the fourth knit pattern forms a second reinforced edge of the second section of mesh. In some embodiments, the warp knit mesh further comprises a void positioned between the first section and the second section, wherein the first reinforced edge of the first section is adjacent the void and wherein the second reinforced edge of the second section is adjacent the void. In some embodiments, the void comprises a slit in the mesh that does not extend to an overall edge of the mesh.

In some embodiments, the warp knit mesh of further comprises one or more tacking yarns connecting the first reinforced edge of the first section to the second reinforced edge of the second section across a void, wherein the one or more tacking yarns are designed to removably connect the first section and the second section. In some embodiments, the void comprises a slit in the mesh that does not extend to an overall edge of the mesh.

In some embodiments, the mesh is designed such that no filament fragments are formed when the one or more tacking yarns are removed.

In some embodiments, the warp knit mesh further comprises one or more first barbs extending from the first reinforced edge connected to one or more second barbs extending from the second reinforced edge so as to connect the first section to the second section across a void.

In some embodiments, the portion of the first knit pattern is a single loop. In some embodiments, the portion of the second knit pattern is a double loop.

In some embodiments, the first reinforced edge is smooth such that it does not have cut ends.

In another example embodiment a method of forming a warp knit mesh is provided. The method comprises: supplying a plurality of warp knit yarns to a warp knit machine; and knitting the plurality of warp knit yarns into a first knit pattern and a second knit pattern. At least a portion of the first knit pattern extends along at least two adjacent courses or wales, and wherein at least a portion of the second knit pattern extends adjacent the first knit pattern along one of the at least two adjacent courses or wales in a parallel manner. The portion of the second knit pattern, as extended, has a higher density of loops or underlaps than the portion of the first knit pattern, as extended, such that the extended portion of the second knit pattern forms a first reinforced edge of the first section of mesh.

In some embodiments, the method further comprises cleaning the warp knit mesh.

In some embodiments, the method further comprises setting the mesh into a final shape. In some embodiments, setting the mesh comprises a thermal process or a heating process.

In another example embodiment, a warp knit mesh is provided. The warp knit mesh comprises: a first section, the first section comprising: a plurality of first warp knit yarns; and a second warp knit yarn disposed at an end of the first section. The plurality of first warp knit yarns each comprise a first knit pattern. The second warp knit yarn comprises a second knit pattern. The second knit pattern, as extended, has a higher density of loops or underlaps than the first knit pattern, as extended, such that the extended second warp knit yarn forms a first reinforced edge for the first section of the mesh.

In some embodiments, the warp knit mesh further comprises a plurality of third warp knit yarns that form nibs or barbs extending past the second warp knit yarn opposite the plurality of first warp knit yarns.

In another example embodiment, a method of forming a warp knit mesh is provided. The method comprises: supplying a plurality of warp knit yarns to a warp knit machine, wherein the plurality of warp knit yarns comprises a plurality of first warp knit yarns and a second warp knit yarn; and knitting the plurality of first warp knit yarns into a first knit pattern and the second warp knit yarn into a second knit pattern, wherein the second knit pattern, as extended, has a higher density of loops or underlaps than the first knit pattern, as extended, such that the extended second warp knit yarn forms a first reinforced edge for a first section of the mesh.

In some embodiments, the plurality of warp knit yarns further comprises at least one third warp knit yarn, wherein the method further comprises knitting the third warp knit yarn into a third knit pattern that, as extended, has a higher density of loops or underlaps than the second knit pattern and the first knit pattern.

In another example embodiment, a warp knit mesh is provided. The warp knit mesh comprises: a first section, the first section comprising: a plurality of first warp knit yarns formed into a first knit pattern and at least one second warp knit yarn. At least a portion of the first knit pattern is designed such that an end warp knit yarn of the plurality of first warp knit yarns crosses from a first wale to a second wale when transitioning between a first course and an adjacent second course such that at least one needle point within the second wale is left uncovered by the portion of the first knit pattern at a knit pattern edge of the portion of the first knit pattern. The at least one second warp knit yarn arranged adjacently to the end warp knit yarn of the plurality of first warp knit yarns and formed into a second knit pattern, wherein the first knit pattern is different than the second knit pattern, wherein at least a portion of the second knit pattern is designed such that the at least one second warp knit yarn forms a section edge for the first section for at least the first course and the second course such that the at least one second warp knit yarn at least loops around the at least one needle point within the second wale that was left uncovered by the portion of the first knit pattern.

In some embodiments, the at least one second warp knit yarn forms the section edge for the first section for at least the first course and the second course by stopping at the second wale without extending to any needle points within the first course or the second course for a further adjacent third wale so as to form a smooth edge without cut ends.

In some embodiments, the at least one second warp knit yarn forms the section edge for the first section for at least the first course and the second course along a further positioned third wale by stopping at the further positioned third wale without extending to any needle points within the first course or the second course for a subsequent further positioned fourth wale so as to form a smooth edge without cut ends, wherein the subsequent further positioned fourth wale is adjacent to the further positioned third wale.

In some embodiments, the first wale is adjacent to the second wale.

In some embodiments, the first section further includes at least one third warp knit yarn that forms at least a portion of a third knit pattern that extends past the portion of the second knit pattern relative to the portion of the first knit pattern, wherein the portion of the third knit pattern comprises nibs or barbs extending past the section edge.

In some embodiments, the warp knit mesh further comprises a second section, wherein the second section comprises: a plurality of second warp knit yarns formed into a third knit pattern and at least one third warp knit yarn. At least a portion of the third knit pattern is designed such that an end warp knit yarn of the plurality of second warp knit yarns crosses from a third wale to a fourth wale when transitioning between the first course and the second course such that at least one needle point within the fourth wale is left uncovered by the portion of the third knit pattern at a knit pattern edge of the portion of the third knit pattern. The at least one third warp knit yarn being arranged adjacently to the end warp knit yarn of the plurality of second warp knit yarns and formed into a fourth knit pattern, wherein the third knit pattern is different than the fourth knit pattern, wherein at least a portion of the fourth knit pattern is designed such that the at least one third warp knit yarn forms a section edge for the second section for at least the first course and the second course such that the at least one third warp knit yarn at least loops around the at least one needle point within the fourth wale that was left uncovered by the portion of the third knit pattern.

In some embodiments, the warp knit mesh further comprises a void positioned between the first section and the second section, wherein the section edge of the first section is adjacent the void and wherein the section edge of the second section is adjacent the void. In some embodiments, the void comprises a slit in the mesh that does not extend to an overall edge of the mesh.

In some embodiments, the warp knit mesh further comprises one or more tacking yarns connecting the section edge of the first section to the section edge of the second section across a void, wherein the one or more tacking yarns are designed to removably connect the first section and the second section. In some embodiments, the void comprises a slit in the mesh that does not extend to an overall edge of the mesh.

In some embodiments, the mesh is designed so as to have smooth edges when the one or more tacking yarns are removed.

In some embodiments, the warp knit mesh further comprises a band section formed of a fifth knit pattern, wherein the first section and the second section extend from the band section, wherein the plurality of warp knit yarns for the first section are a plurality of first warp knit yarns, and wherein the plurality of first warp knit yarns and the plurality of second warp knit yarns transition into the fifth knit pattern when forming the band section. In some embodiments, the band section is further formed of one or more tacking warp knit yarns extending between the first section and the second section, wherein the one or more tacking yarns reengage into the fifth knit pattern when in the band section, wherein the one or more tacking yarns are removable with respect to the first section or the second section.

In another example embodiment a method of forming a warp knit mesh is provided. The method comprises: supplying a plurality of warp knit yarns to a warp knit machine, the plurality of warp knit yarns comprising at least a plurality of first warp knit yarns and at least one second warp knit yarn, wherein the at least one second warp knit yarn is arranged adjacently to an end warp knit yarn of the plurality of first warp knit yarns; and knitting the plurality of warp knit yarns into a first knit pattern and a second knit pattern for a first section of the mesh. At least a portion of the first knit pattern is designed such that the end warp knit yarn of the plurality of first warp knit yarns crosses from a first wale to a second wale when transitioning between a first course and an adjacent second course such that at least one needle point within the second wale is left uncovered by the portion of the first knit pattern at a knit pattern edge of the portion of the first knit pattern. At least a portion of the second knit pattern is designed such that the at least one second warp knit yarn forms a section edge for the first section for at least the first course and the second course such that the at least one second warp knit yarn at least loops around the at least one needle point within the second wale that was left uncovered by the portion of the first knit pattern. The first knit pattern is different than the second knit pattern.

In some embodiments, the at least one second warp knit yarn forms the section edge for the first section for at least the first course and the second course by stopping at the second wale without extending to any needle points within the first course or the second course for a further adjacent third wale so as to form a smooth edge without cut yarns.

In some embodiments, the at least one second warp knit yarn forms the section edge for the first section for at least the first course and the second course along a further positioned third wale by stopping at the further positioned third wale without extending to any needle points within the first course or the second course for a subsequent further positioned fourth wale so as to form a smooth edge without cut yarns, wherein the subsequent further positioned fourth wale is adjacent to the further positioned third wale.

In some embodiments, the first wale is adjacent to the second wale.

In some embodiments, the plurality of warp knit yarns further comprises at least a plurality of third warp knit yarns and at least one fourth warp knit yarn, wherein the at least one fourth warp knit yarn is arranged adjacently to an end warp knit yarn of the plurality of third warp knit yarns, wherein the method further comprises knitting the plurality of warp knit yarns into a third knit pattern and a fourth knit pattern for a second section of the mesh. At least a portion of the third knit pattern is designed such that the end warp knit yarn of the plurality of third warp knit yarns crosses from a third wale to a fourth wale when transitioning between the first course and the second course such that at least one needle point within the fourth wale is left uncovered by the portion of the third knit pattern at a knit pattern edge of the portion of the third knit pattern. At least a portion of the fourth knit pattern is designed such that the at least one fourth warp knit yarn forms a section edge for the second section for at least the first course and the second course such that the at least one third warp knit yarn at least loops around the at least one needle point within the fourth wale that was left uncovered by the portion of the third knit pattern. The third knit pattern is different than the fourth knit pattern.

In some embodiments, knitting the plurality of warp knit yarns into the third knit pattern and the fourth knit pattern for the second section of the mesh occurs simultaneously with formation of the first section of the mesh.

In another example embodiment, a warp knit mesh is provided. The warp knit mesh comprises: a first section, the first section comprising: a plurality of first warp knit yarns formed into a first knit pattern, wherein at least a portion of the first knit pattern is designed such that an end warp knit yarn of the plurality of first warp knit yarns crosses from a first wale to a second wale when transitioning between a first course and an adjacent second course; and at least one second warp knit yarn arranged adjacently to the end warp knit yarn of the plurality of first warp knit yarns and formed into a second knit pattern, wherein the first knit pattern is different than the second knit pattern, wherein at least a portion of the second knit pattern is designed such that the at least one second warp knit yarn forms a section edge for the first section for at least the first course and the second course by either (i) stopping at the second wale without extending to any needle points within the first course or the second course for a further adjacent third wale, or (ii) forming the section edge for the first section for at least the first course and the second course along a further positioned third wale by stopping at the further positioned third wale without extending to any needle points within the first course or the second course for a subsequent further positioned fourth wale, wherein the subsequent further positioned fourth wale is adjacent to the further positioned third wale.

In some embodiments, the section edge is smooth without cut yarns.

In another example embodiment, a warp knit mesh is provided. The warp knit mesh comprises: a band comprising a plurality of warp knit yarns defining at least a first knit pattern, wherein the plurality of warp knit yarns comprises at least a first set of warp knit yarns and a second set of warp knit yarns, wherein the first set of warp knit yarns are different than the second set of warp knit yarns; and a plurality of strips extending from the band, wherein the plurality of strips includes at least a first strip and a second strip. The first strip is formed of the first set of warp knit yarns, wherein the first set of warp knit yarns define a second knit pattern within the first strip that is different than the first knit pattern. The second strip extends from the band in a parallel fashion to the first strip, the second strip formed of the second set of warp knit yarns, wherein the second set of warp knit yarns define the second knit pattern. The first strip and the second strip are separated by a gap therebetween, wherein the gap corresponds to at least one pick position between an edge of the first strip and an edge of the second strip.

In some embodiments, the plurality of warp knit yarns forming the band further comprises one or more tacking warp knit yarns, wherein the one or more tacking warp knit yarns, the first set of warp knit yarns, and the second set of warp knit yarns together form the first knit pattern, and wherein the one or more tacking warp knit yarns extend within the gap and are removable.

In some embodiments, the one or more tacking warp knit yarns are reengaged into the first knit pattern within the band.

In some embodiments, the first set of warp knit yarns transition between the first knit pattern and the second knit pattern between a first course and a second course.

In some embodiments, the mesh is designed such that there are no cut yarns between the first strip and the second strip.

In some embodiments, the second knit pattern comprises an edge knit pattern and a body knit pattern, wherein the edge knit pattern and the body knit pattern are different. In some embodiments, the first strip comprises a first edge, wherein the second strip comprises a second edge, wherein the first edge and the second edge comprise the edge knit pattern, wherein the edge knit pattern is continuous along a length of the first strip or the second strip, and wherein the edge knit pattern is smooth such that there are no cut yarns. In some embodiments, the edge knit pattern comprises single loops and underlaps along the length of the first strip or the second strip. In some embodiments, the edge knit pattern comprises at least one double loop, overlap, or underlap along the length of the first strip or the second strip.

In another example embodiment, a method of forming a knit structure is provided. The method comprises: knitting a plurality of warp knit yarns in a first knit pattern to form a band within the knit structure, wherein each of the warp knit yarns within the plurality of warp knit yarns is used in the first knit pattern; and transitioning the plurality of warp knit yarns from the first knit pattern to a second knit pattern forming at least a first strip, a second strip, and a tacking yarn positioned therebetween, wherein, in the second knit pattern, at least one warp knit yarn of the plurality of warp knit yarns becomes the tacking yarn.

In some embodiments, the first strip is formed of a first set of the plurality of warp knit yarns, and the second strip is formed of a second set of the plurality of warp knit yarns.

In some embodiments, the second knit pattern comprises an edge knit pattern and a body knit pattern, wherein the edge knit pattern and the body knit pattern are different, wherein the edge knit pattern is smooth so as to define no cut yarns.

In some embodiments, the first strip comprises a first edge, wherein the second strip comprises a second edge, wherein the first edge and the second edge comprise the edge knit pattern, wherein the edge knit pattern is continuous along a length of the first strip or the second strip.

In some embodiments, the method further comprises removing the tacking yarn between the first strip and the second strip, wherein the tacking yarn remains knit within the first knit pattern.

In some embodiments, the knit structure is designed such that no cut yarns are formed between the first strip and the second strip.

In some embodiments, the method further comprises cleaning the knit structure.

In some embodiments, the method further comprises setting the knit structure into a final shape.

In some embodiments, setting the knit structure comprises a thermal process or a heating process.

In another example embodiment, a warp knit mesh is provided. The warp knit mesh comprises: a first section, the first section comprising: a plurality of warp knit yarns extending in side-by side relation, wherein the plurality of warp knit yarns comprises at least a first set of warp knit yarns, a second set of warp knit yarns, and one or more tacking warp knit yarns that together form a first knit pattern within the first section; and a second section, the second section comprising: a first strip defined by the first set of the plurality of warp knit yarns, wherein the first set of the plurality of warp knit yarns extend in side-by-side relation in a second knit pattern that is different than the first knit pattern; a second strip defined by the second set of the plurality of warp knit yarns, wherein the second set of the plurality of warp knit yarns extend in side-by side relation in the second knit pattern; and a tacking section defined by the one or more tacking warp knit yarn formed into a third knit pattern that is different than the first knit pattern and the second knit pattern. The first set of the plurality of warp knit yarns, the second set of the plurality of warp knit yarns, and the one or more tacking warp knit yarns transition together from the first knit pattern to the respective second knit patterns and third knit pattern across one or more courses.

In some embodiments, the one or more tacking warp knit yarn is removable in relation to the first strip and the second strip so as to form a gap between the first strip and the second strip.

### BRIEF DESCRIPTION OF THE DRAWINGS

Having thus described the invention in general terms, reference will now be made to the accompanying drawings, which are not necessarily drawn to scale, and wherein:

FIG. 1 illustrates an example mesh, in accordance with some embodiments discussed herein;

FIGS. 2A-D illustrate example mesh without reinforced edges;

FIG. 3A illustrates an example mesh with an example reinforced edge, in accordance with some embodiments discussed herein;

FIG. 3B illustrates an example mesh with a second example reinforced edge, in accordance with some embodiments discussed herein;

FIG. 3C illustrates an example mesh with voids formed in the mesh, in accordance with some embodiments discussed herein;

FIG. 4 illustrates an example warp knitting pattern, in accordance with some embodiments discussed herein;

FIG. 5A illustrates an example stitch notation for single loop mesh, in accordance with some embodiments discussed herein;

FIG. 5B illustrates an example stitch resulting from the stitch notation illustrated in FIG. 5A, in accordance with some embodiments discussed herein;

FIG. 6A illustrates an example stitch notation for double loop mesh, in accordance with some embodiments discussed herein;

FIG. 6B illustrates an example stitch resulting from the stitch notation illustrated in FIG. 6A, in accordance with some embodiments discussed herein;

FIGS. 7A-E illustrate example joins between two example reinforced edges, in accordance with some embodiments discussed herein;

FIG. 8A illustrates an example reinforced edge of the mesh illustrated in FIG. 7B, wherein tack yarns have been severed to form the mesh edge, in accordance with some embodiments discussed herein;

FIG. 8B illustrates an example reinforced edge of the mesh with barbed edges, in accordance with some embodiments discussed herein;

FIG. 9A illustrates an example mesh without a reinforced edge in a stretched position, in accordance with some embodiments discussed herein;

FIGS. 9B-C illustrate example mesh with a reinforced edge in a stretched position, in accordance with some embodiments discussed herein;

FIG. 10A illustrates example stitch notation for forming a knit pattern of a reinforced edge perpendicular to the machine direction, in accordance with some embodiments discussed herein;

FIG. 10B illustrates an example stitch notation for forming a knit pattern of a reinforced edge in the machine direction, in accordance with some embodiments discussed herein;

FIGS. 11A-B illustrate example stitch notations for forming example reinforced edges, in accordance with some embodiments discussed herein;

FIG. 12A-C illustrate example stitch notations for forming an example warp knit mesh, in accordance with some embodiments discussed herein;

FIG. 13 illustrates an example stitch notation for sections of a warp knit mesh, in accordance with some embodiments discussed herein;

FIG. 14A illustrates an example stitch notation for forming an example warp knit mesh, in accordance with some embodiments discussed herein;

FIG. 14B illustrates an example stitch notation for forming another example warp knit mesh, in accordance with some embodiments discussed herein; and

FIG. 15 shows a flowchart of an example method of forming a mesh, in accordance with some embodiments discussed herein.

### DETAILED DESCRIPTION OF THE INVENTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

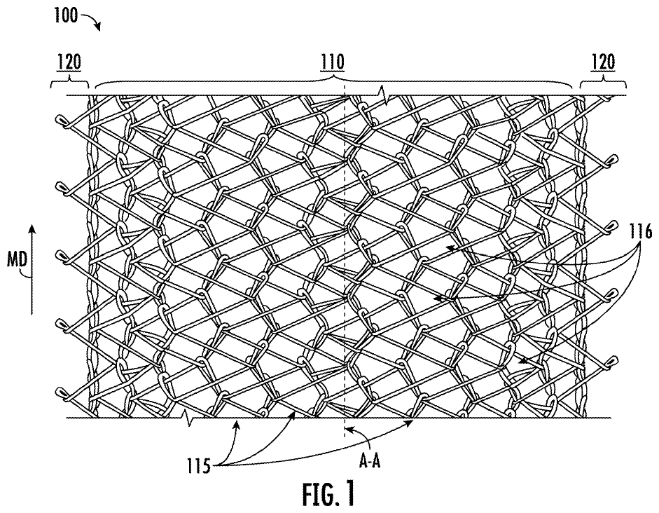

FIG. 1 illustrates an example mesh 100 comprising reinforced edges 120 extending on opposing sides of a body 110 of the mesh 100. The body 110 comprises a plurality of warp knit yarns 115 which are knit together in a first knit pattern as will be described herein. The reinforced edges 120 are formed from a second knit pattern, which is denser than the first knit pattern. In the illustrated embodiment, the reinforced edges 120 are formed in the machine direction which extends vertically (e.g., from the bottom of the mesh 100 to the top of the mesh 100), although they could (additionally or alternatively) be formed in the cross direction.

In some embodiments, the warp knit yarn 115 may be any textile strand, including any filament, filament yarn (including single filament or multi-filament yarns) or spun yarn.

Likewise, the warp knit yarn 115 may be of synthetic or natural material. In some embodiments, the warp knit yarn may be a synthetic biocompatible material, such as, but not limited to, polyester, polypropylene, polyethylene, polytetrafluorethylene (PTFE), polyglactin, polyglycolic acid, trimethylene carbonate, poly-4-hydroxy butyrate (P4HB), polyglycolide, polyactide, and trimethylene carbonate (TMC). In other embodiments, the warp knit yarn 115 may comprise a combination of synthetic and/or natural materials, for example silk.

In some embodiments, the warp knit yarn 115 may be resorbable, while in other embodiments the warp knit yarn 115 may be non-resorbable. In some embodiments, more than one type of warp knit yarn 115 may be used within the mesh 100. In this regard, a first set of warp knit yarn may be formed from a first yarn, and a second set of warp knit yarn may be formed from a second yarn. In some embodiments, the first and second yarns may be different sizes (e.g., thickness), display different physical properties (e.g., elasticity), comprise different filaments, and/or comprise different synthetic or natural materials.

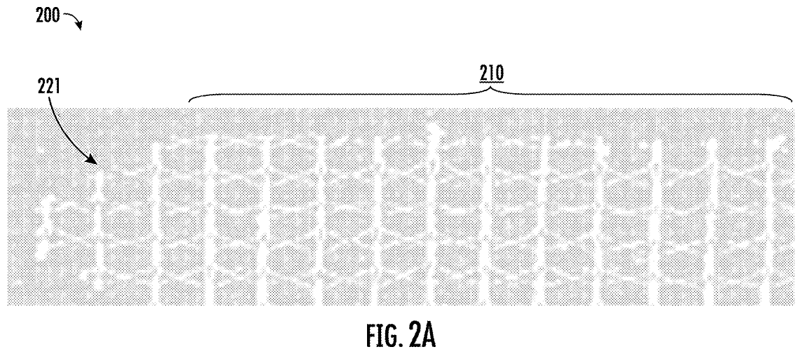

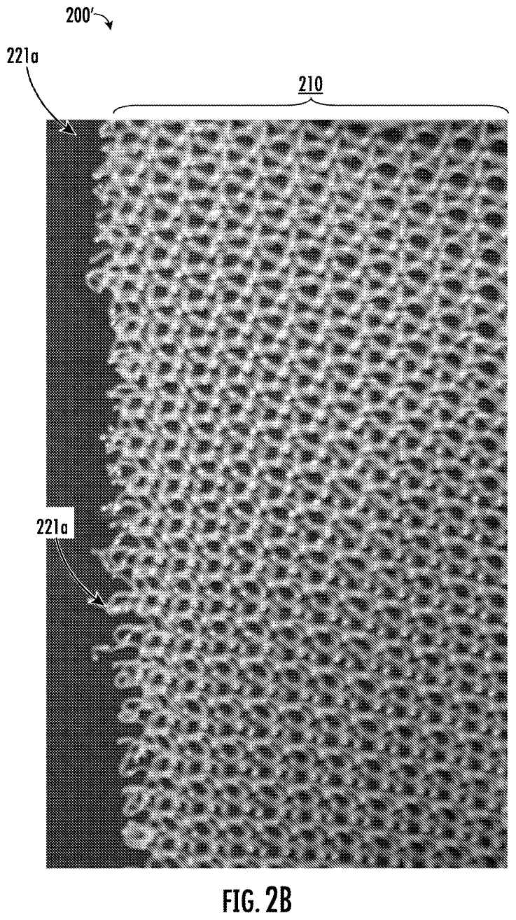

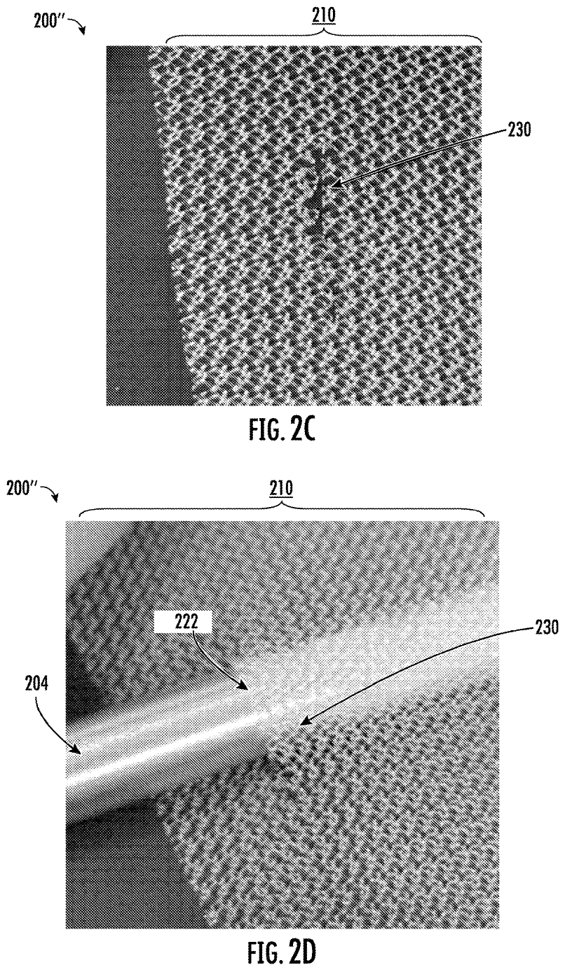

The mesh 100 may be a knitted mesh fabric defining openings or pores 116 throughout. As shown in the illustrated mesh 100, the knit pattern formed within the body 110 of the mesh 100 is formed of the first knit pattern including diagonal underlaps and loops that are intertwined with one another. The first knit pattern, when used as the sole knit pattern in a mesh, may cause the mesh to be hard to cut. In this regard, in the illustrated embodiment, cutting through the center of the mesh 100 (e.g., along line A-A) causes loose edges of the mesh, resulting in fraying and potentially unraveling. To explain, the linear cut along line A-A causes the plurality of warp knit yarns 115 to be cut at different portions along the stitch. As each wale within the body comprises multiple yarns of the plurality of warp knit yarns 115, the edges may fray and unravel as depicted in FIG. 2A.

FIGS. 2A-D illustrate various mesh 200, 200′, 200″ without reinforced edges (e.g., 120 FIG. 1) after being cut into a discrete section. FIG. 2A illustrates an example mesh 200 with an un-reinforced edge 221. As illustrated, the mesh 200 does not have stability in the un-reinforced edge 221 and is prone to unraveling when exposed to tension or friction. Likewise, FIG. 2B illustrates an edge 221 formed by a cut through a body 210 of the mesh 200′, to form, for example, a discrete section of mesh. As the photograph illustrates, the edge 221 defines loose ends which may easily unravel when exposed to friction or objects. Although the mesh 200, 200′ may be cut with a laser or similar heat cut to attempt to prevent unraveling, by melting the edges, loose ends 221a may still prevail and cause fragments and unraveling of the mesh 200. Further, such melt-formed edges are stiff and abrasive and undesirable for use in many circumstances.

FIGS. 2C-D illustrate a void 230 (e.g., slit) through an example mesh 200″. In some embodiments, the void 230 may be formed to allow an object 204 to pass through the mesh 200, for example, to position a catheter, textile component, or other lumen. In this regard, positioning the object 204 through the void 230 may create friction between the object 204 and either edge 222 of the void 230. In this regard, the lack of reinforcement about the void 230 may cause the plurality of warp knit yards to unravel from the body 210 of the mesh, rather than only the edges 221. Notably, while the illustrated embodiment shows an object 204, any object (e.g., a needle, a suture, another mesh, a medical device, etc.) may be envisioned to pass through the void (e.g., slit).

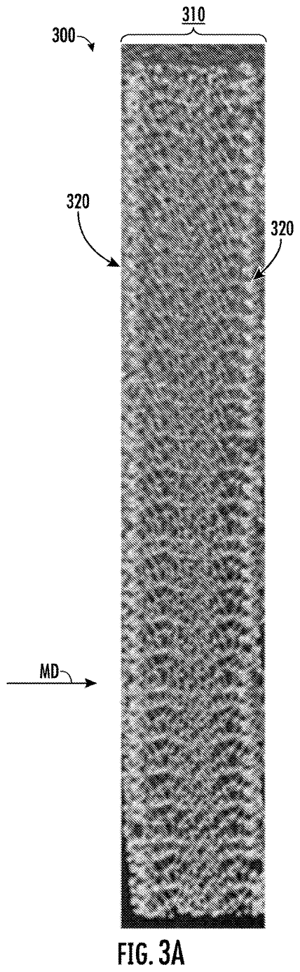

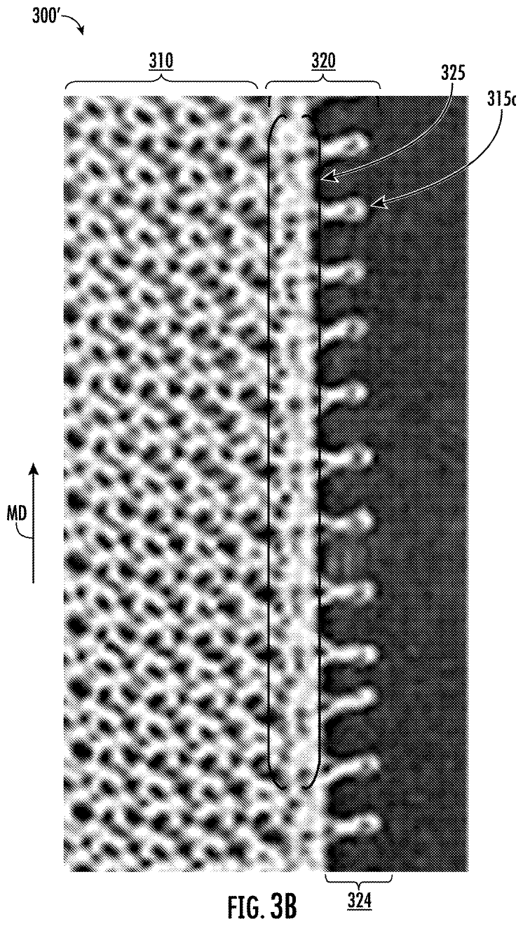

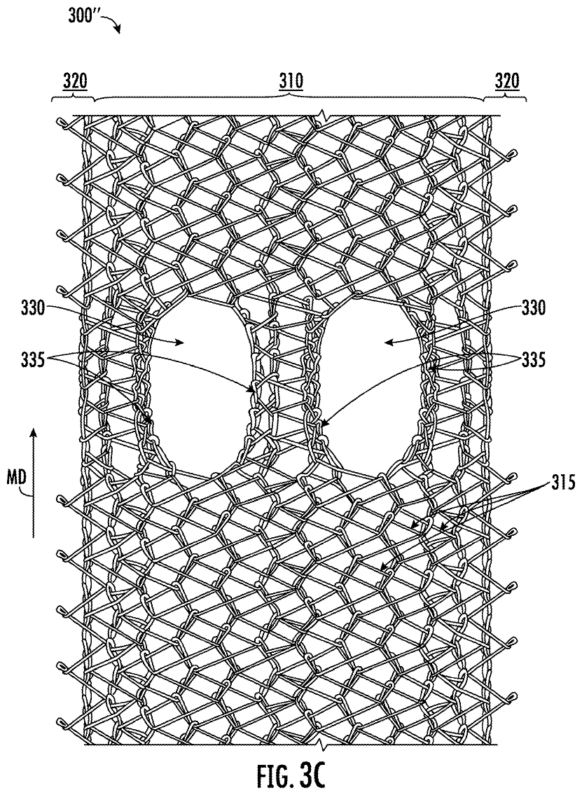

Thus, to prevent the mesh from unraveling and/or fraying, such as due to end placement of the mesh (e.g., rubbing during installation and/or at the final destination) or due to cutting the mesh to size, the mesh may be formed with reinforced edges, as illustrated in FIGS. 3A-C. FIG. 3A illustrates a mesh 300 comprising a body 310 and reinforced edges 320. In some embodiments, the body 310 may define a first knit pattern, and the reinforced edges 320 may comprise a second knit pattern. In some embodiments, the first knit pattern, once extended, may include a lower density of loops or underlaps than the second knit pattern, once extended. In some embodiments, additional knit patterns can be further applied, forming different levels of edges (e.g., a third knit pattern can be applied that has even a higher density of loops or underlaps, once extended). In some embodiments, different patterns of knit patterns and densities of loops or underlaps can be utilized to form the desired reinforced edge. Along similar lines, in some embodiments, the second knit pattern may comprise a different amount of yarn per stitch than the first knit pattern. Notably, in some embodiments, depending on the configuration and/or desired characteristics of the mesh, the second knit pattern forming the reinforced edge may have more or less loops or more or less underlaps than the first knit pattern forming a main pattern of the mesh. In this regard, however, the second knit pattern is designed to provide reinforcement and some increased rigidity with respect to the first knit pattern. Notably, however, the reinforced edge is smooth and free of cut ends.

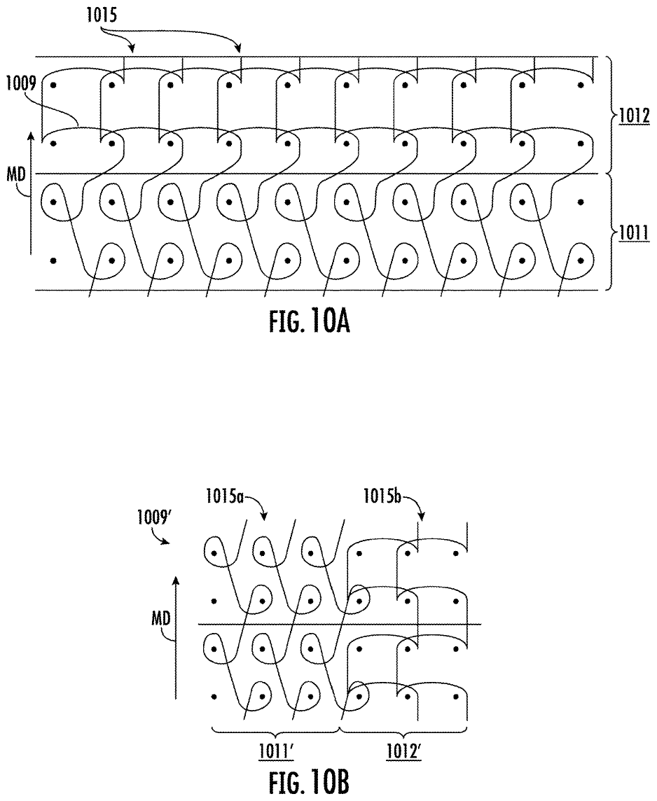

In some embodiments, the reinforced edges 320 may be formed in the machine direction, while additionally or alternatively, in other embodiments the reinforced edges 320 may be formed perpendicular to the machine direction. For example, FIGS. 10A-B illustrate example stitch notations for forming knit patterns for reinforced edges in the machine direction (e.g., 1021 in FIG. 10B) and perpendicular to the machine direction (e.g., 1021 in FIG. 10A).

FIG. 10A illustrates a stitch notation 1009 for forming a first knit pattern 1011 and a second knit pattern 1012 perpendicular to the machine direction (e.g., along course(s) of the mesh). The stitch notation 1009 relies on a plurality of yarns 1015 extending in side-by-side relation. As illustrated, the stitches formed in the top two courses form the second knit pattern 1012, while the stitches formed below the top two courses (e.g., adjacent in a parallel manner) may comprise a first knit pattern 1011. In this regard, the plurality of yarns 1015 are formed in the second knit pattern 1012 for a first number of courses and transition to the first knit pattern 1011 for a second number of courses.

In contrast, in some embodiments, such as illustrated in the stitch notation 1009′ shown in FIG. 10B, the knit patterns may be formed in the machine direction. In this regard, plurality of yarns, may comprise a first yarn(s) 1015a and a second yarn(s) 1015b. In some embodiments, the first yarn(s) 1015a may be formed in a first knit pattern 1011′ extending along the wales, and the second yarn(s) 1015b may be formed in a second knit pattern 1012′ extending adjacently and in a parallel manner along the wales. Thus, the wales which comprise the first knit pattern 1011′ are distinct from the wales which comprise the second knit pattern 1012′.

In some embodiments, such as illustrated in FIG. 3B, a mesh 300′ may define a reinforced edge 320 comprising two sections, a reinforced section 325 and a nibbed or barbed section 324. In this regard, the reinforced section 325 may be formed with a second warp knit yarn, and the nibbed or barbed section 324 may be formed with a third warp knit yarn adjacent the second warp knit yarn. To explain, the plurality of first warp knit yarns may be formed in a first knit pattern to form the body 310, the second warp knit yarn may be formed in a second knit pattern to form the reinforced section 325, and the third warp knit yarn 315c may be formed in a third knit pattern to form the nibs (such as shown in the nibbed or barbed section 324). Notably, the illustrated nibs can be formed into barbs by cutting proximate a tip of the nib to form two barbs. In the illustrated embodiment, the reinforced edge 320 is formed in the machine direction, similar to FIG. 10B. In some embodiments, the third warp knit yarn and the third knit pattern may be the same (e.g., type, size, material) as the first warp knit yarn and the first knit pattern, while in other embodiments the third warp knit yarn and third knit pattern may be different than the first warp knit yarn and first pattern. In some embodiments, the third warp knit yarn may be an extension of the first warp knit yarn (e.g., a part of the body 310). In this regard, the reinforced section 325 may be formed within the body 310 of the mesh 300.

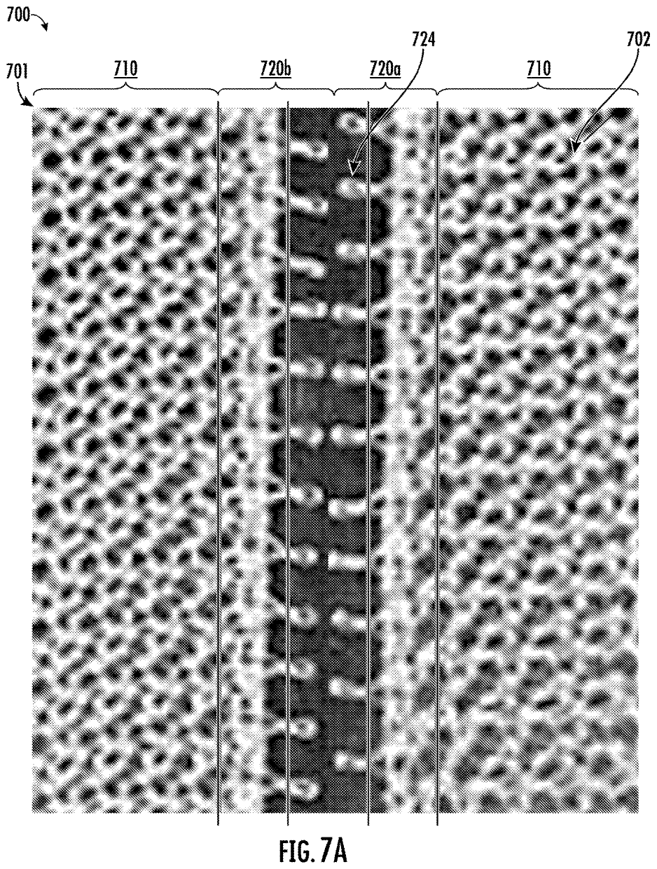

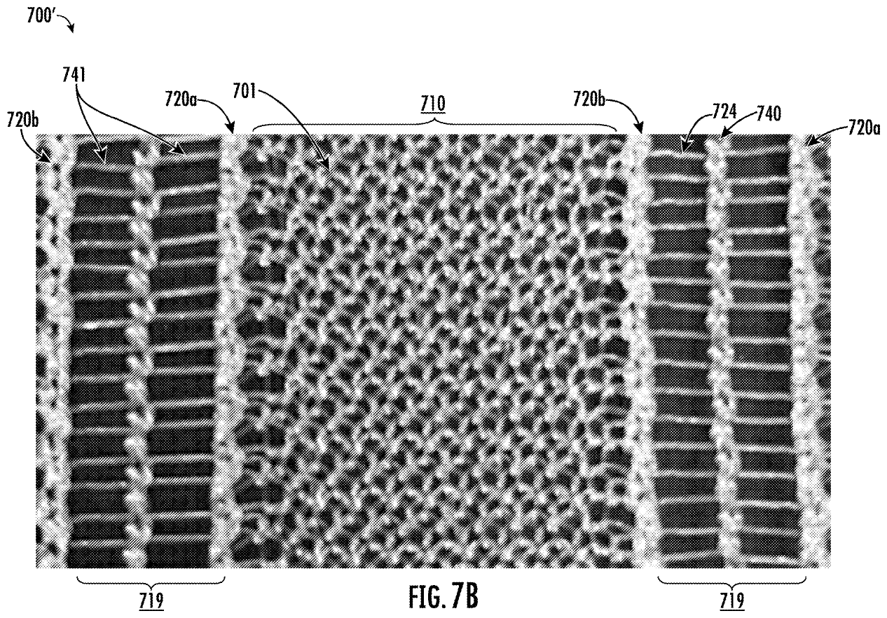

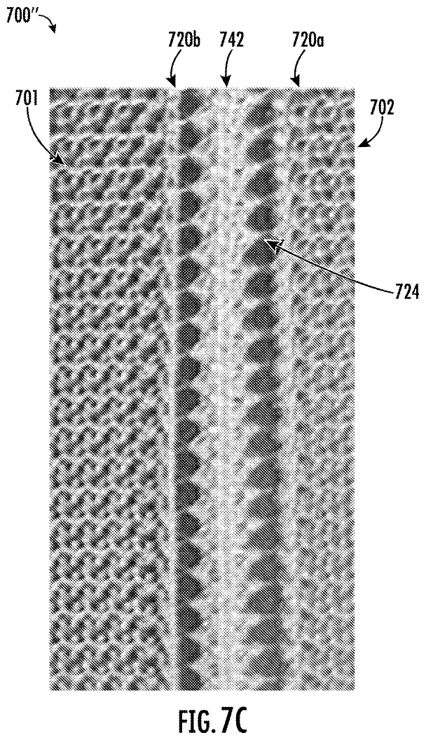

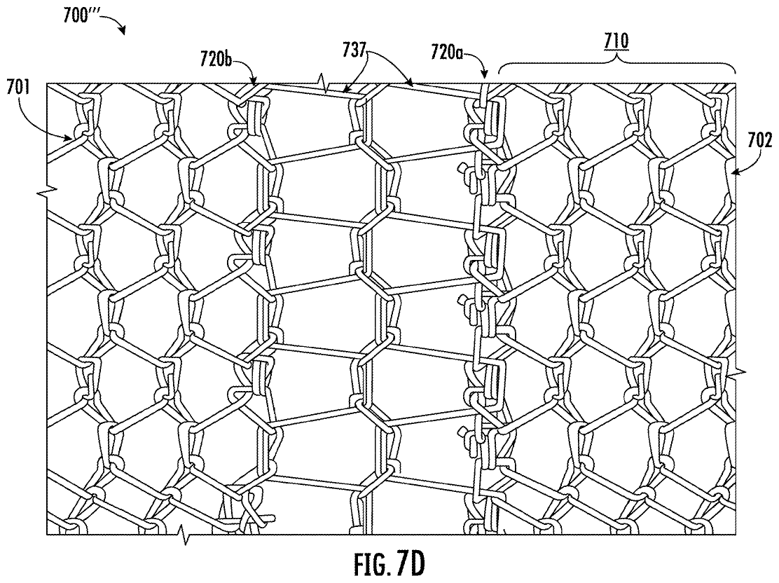

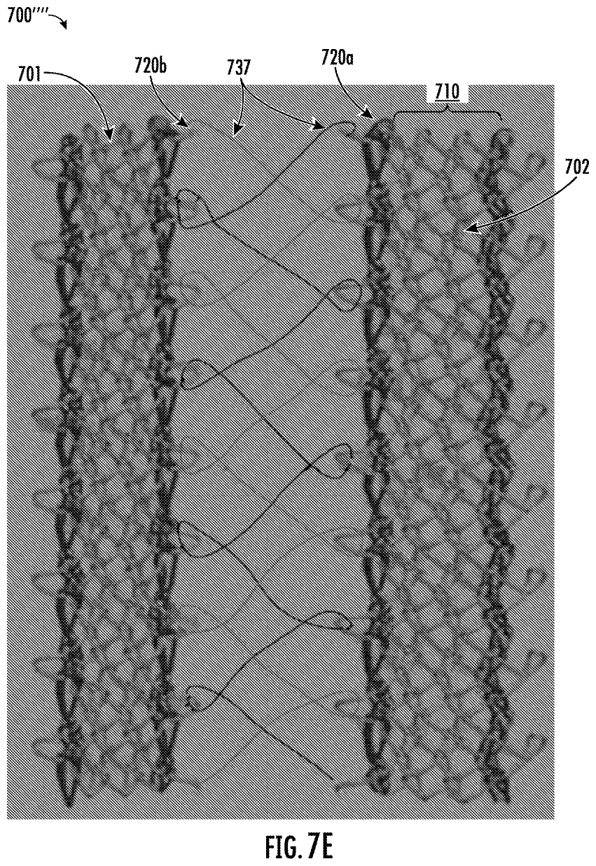

In some embodiments, such as illustrated in FIG. 7C, one or more tacking stitch(es) may be positioned within the nibbed or barbed section 324 to connect discrete sections of the mesh 300′. In some embodiments, the tacking stitches may be formed with a fourth warp knit yarn, that may be different than the first, second, and/or third warp knit yarns, and in some embodiments may be the same as one or more of the first, second, and third warp knit yarns. In some embodiments, however, the mesh 300′ may be formed with the nibbed or barbed section 324 without a tacking stitch. Notably, having the nibbed or barbed section 324 extend from the reinforced section 325 provides barbs that aid in part cohesion and also minimize accidental de-threading or unraveling of the mesh 300′.

FIG. 3C illustrates an example mesh 300″ comprising voids 330 formed within the body 310 of the mesh 300″. Each of the voids 330 is defined by the plurality of warp knit yarns 315 being directed into a reinforced void edge 335 about the void 330. In some embodiments, the reinforced void edge 335 may comprise the same second knit pattern as the reinforced edge 320 as will be discussed more herein. Thus, the reinforced void edge 335 prevents fraying and/or unraveling of the mesh due to the presence of the void 330, such as due to interaction with other objects, as detailed herein. Notably, the size of the void 330 may be customized to the desired specifications. In some embodiments, the void 330 may be spanned by one or more tacking stitches that may be designed to, for example, be cut and/or pulled to form the void after manufacturing. In such an example, the void is easily formed on demand. In some embodiments, the reinforced void edge 335 may include a nibbed or barbed section, such as illustrated in FIG. 3B.

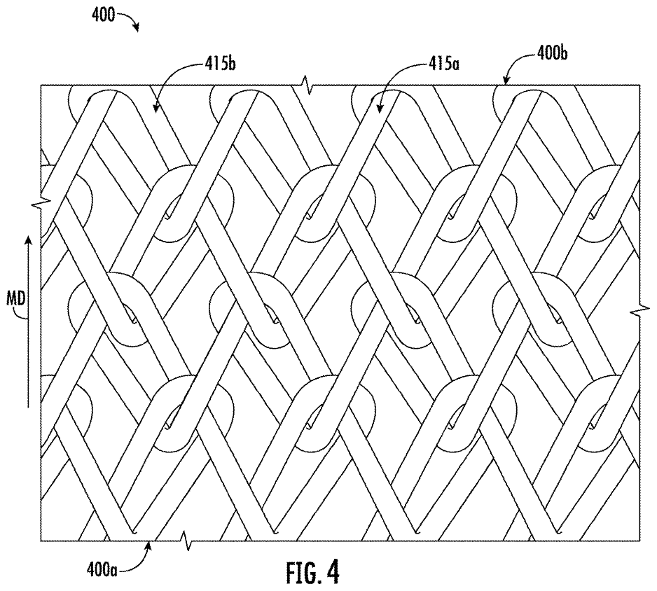

FIG. 4 illustrates an example warp knit mesh 400 and the individual yarns which make up the body of the mesh 400. In the body of the mesh 400, each individual yarn 415a, 415b of the plurality of warp knit yarns 415 is warp knitted. In warp knitting, each yarn 415a, 415b forms wales that are not vertically aligned, but rather extend diagonally between each course (e.g., the horizontal stitch). As the mesh 400 is formed in the machine direction, for example, from a bottom 400a to a top 400b of the illustrated mesh 400, the individual yarns 415a, 415b are not always wrapped around the same needle, creating the diagonal pattern.

As illustrated, due to the warp knitting pattern, as described with reference to FIG. 1, when the mesh 400 is severed diagonally or vertically (e.g., in the machine direction) the cut may create fragments (e.g., cut yarn portions) of the wale loops which may be retained by the mesh 400.

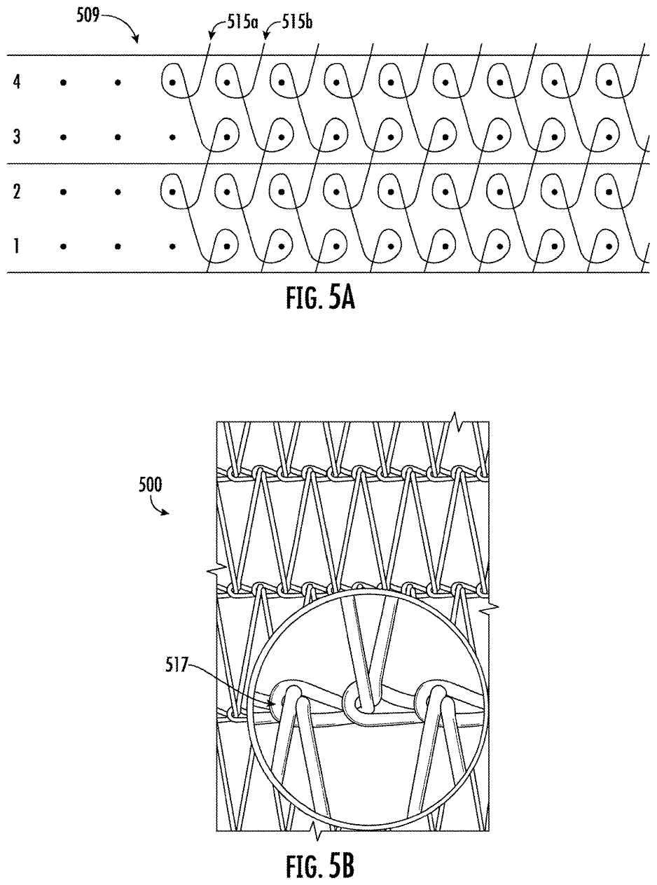

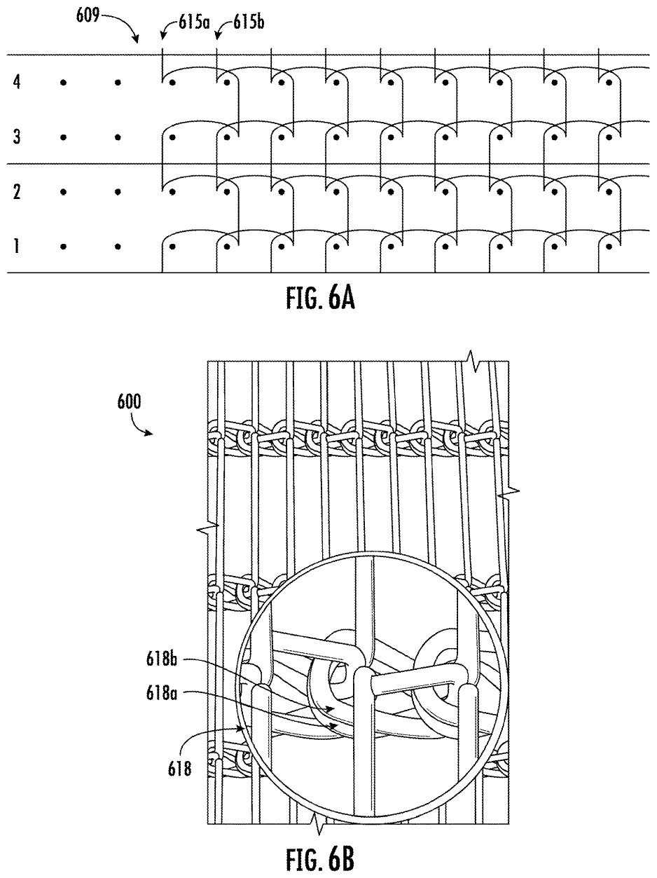

To explain further, FIGS. 5A-6B illustrate example stitch patterns for warp knitting, and the resulting structure. FIGS. 5A-B illustrate a single loop stitch notation pattern 509 to create a warp knit 500 with a single loop knit. Each individual yarn 515a, 515b corresponds to a guide bar. The guide bar is configured to lead the individual yarn 515a, 515b to alternate back and forth between two adjacent needle rows, thereby forming the diagonal pattern shown in FIG. 4. Further, in the single loop pattern each individual yarn 515a, 515b is wrapped around each needle once, thereby forming a single loop join 517, such as illustrated in FIG. 5B. In some embodiments, the first knit pattern may be a single loop pattern as illustrated.

Another stitch pattern may be the double loop, such as illustrated in FIGS. 6A-B. FIG. 6A illustrates a stitch notation 609 to form the double loop. To form the double loop each individual yarn 615a, 615b is positioned within a guide bar, and at each course (e.g., listed 1, 2, 3, 4), the individual yarn 615a, 615b is looped around two adjacent needles, forming a double loop join 618. Therefore, each double loop join 618 comprises two loops 618a, 618b corresponding to two different individual yarns. This stitch pattern increases durability and may create a straight wale or a straight course line, in comparison to the single loop. In some embodiments, the second knit pattern may be a double loop. In some embodiments, the second knit pattern may comprise a mixture of single loops, double loops, overlaps, or underlaps (e.g., two adjacent double loops followed by a single loop, adjacent overlaps, adjacent underlaps, two adjacent double loops followed by two adjacent single loops, or any other pattern).

Utilizing the combinations of single loop, double loop, overlap, underlap, among other stitches, mesh may be formed with reinforced edges, defined voids, and severance point(s) to create a mesh which does not fray upon cutting or when exposed to friction. In this regard, the reinforced edges may be smooth and free from cut yarns.

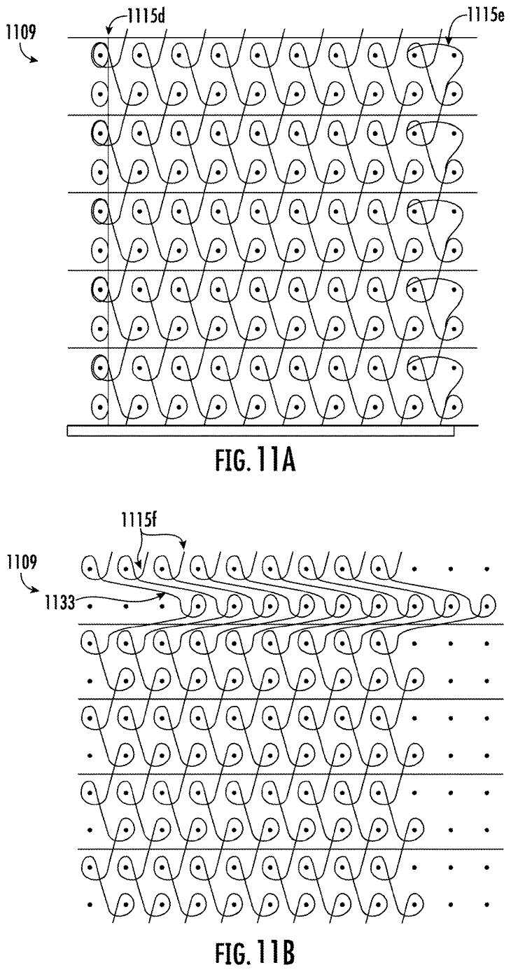

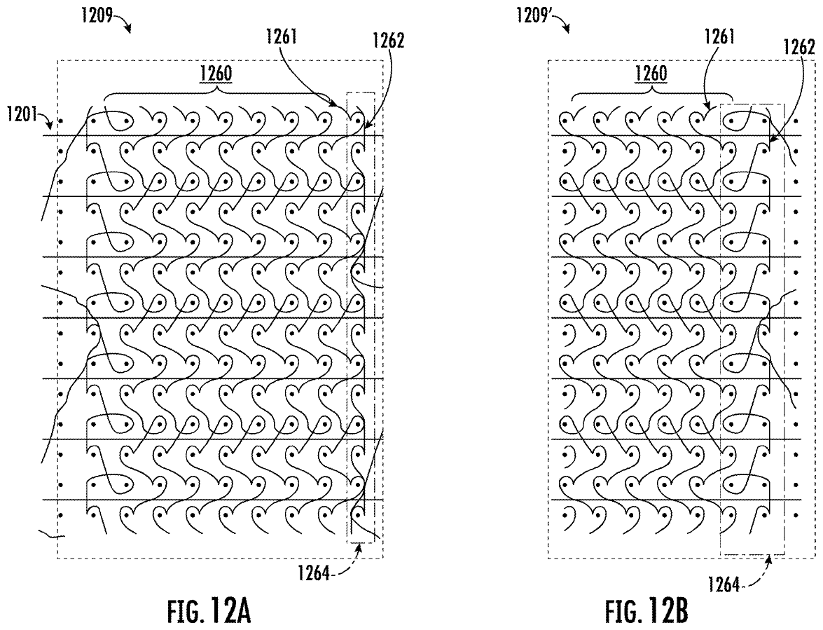

In some embodiments, to define discrete sections within the mesh, or to form voids within the mesh, the reinforced edges of the mesh may be joined, such as illustrated in FIGS. 7A-E. FIG. 7A illustrates a first mesh 700 joining a first section 701 and a second section 702 of the mesh. Each of the segments may define a first reinforced edge 720a and a second reinforced edge 720b, and a body 710 therebetween. In some embodiments, the first reinforced edge 720a may be formed by a first warp knit yarn, and the second reinforced edge 720b may be formed by a second warp knit yarn. The body 710 of the mesh may be formed by a plurality of warp knit yarns according to a different knit pattern than the first reinforced edge 720a and the second reinforced edge 720b. Although illustrated as the same pattern, in some embodiments, various different reinforced edge patterns may be used for one or more of the reinforced edges. Some other example reinforced edge patterns are illustrated in FIGS. 11A-B. With reference to FIG. 11A, an example reinforced edge may comprise a fourth set of warp yarns 1115d configured to recover stitches along the wale direction. Notably, in the illustrated embodiment, the fourth set of warp yarns 1115d may be configured to not cross into an adjacent wale so as to create a linear reinforcement. FIG. 11A also illustrates another example reinforced edge using a fifth set of warp yarns 1115e that are configured to cross more than one wale in a linear pattern. With reference to FIG. 11B, another example reinforced edge is shown in the direction perpendicular to the machine direction. In the illustrated embodiment, a sixth set of warp yarns 1115f comprise underlaps 1133 that cross more than one wale. Notably, other reinforced edges may include double loops, single loops, other types of loops, underlaps, overlaps and/or other features that work together to form a reinforced edge.

Returning to FIG. 7A, in some embodiments, the first reinforced edge 720a and the second reinforced edge 720b comprise a reinforced section (e.g., 325 FIG. 3B) and a nibbed or barbed section 724 extending from the first reinforced edge 720a and the second reinforced edge 720b. In some embodiments, the nibbed or barbed sections 724 can be joined, such as via a chain stitch or similar. In this regard, thereafter, the connection of the nibs 724 may be severed to yield a two distinct mesh sections, each with fray resistant edges.

FIG. 7B illustrates a second mesh 700′ with joining sections 719 between three different sections. The body 710 of the first section 701 may extend between the first reinforced edge 720a to the second reinforced edge 720b. As discussed, the body 710 may comprise a first knit pattern and the first reinforced edge 720a and the second reinforced edge 720b may be comprise a second knit pattern (although they could have different patterns from each other).

To join the adjacent sections (or edges within a section) each of the first reinforced edge 720a and the second reinforced edge 720b may use extensions 741 to connect the respective reinforced edge to an anchor 740. In some embodiments, the anchor 740 may comprise the second knit pattern, while in other embodiments the anchor 740 may comprise a third knit pattern, different than, for example, both the first knit pattern and the second knit pattern. In some embodiments, the anchor 740 may be used to retain the structure of the section 701 during knitting, and prior to final shaping. In this regard, the anchor 740 may be used when the width between the sections, or the width of the void is large, and the structure of the mesh needs to be retained.

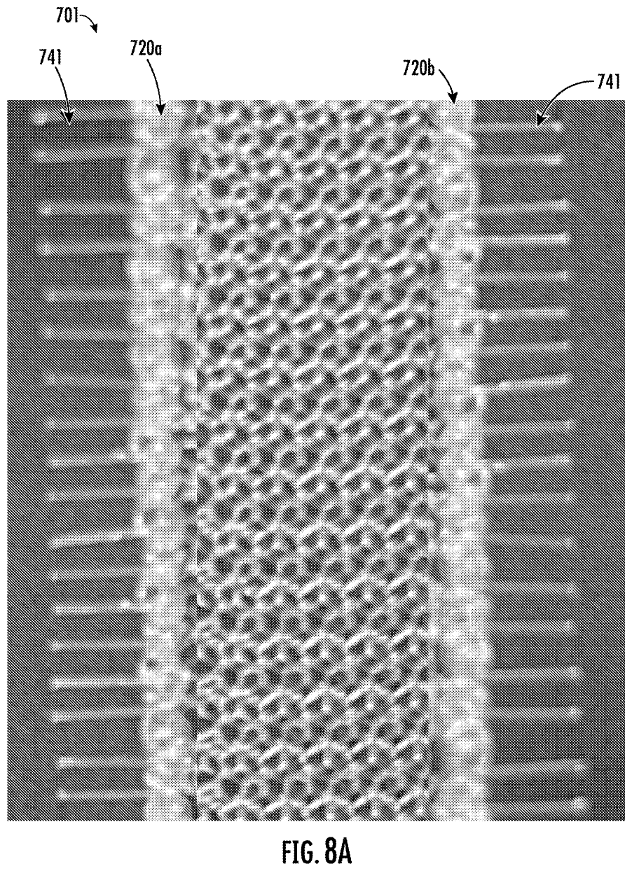

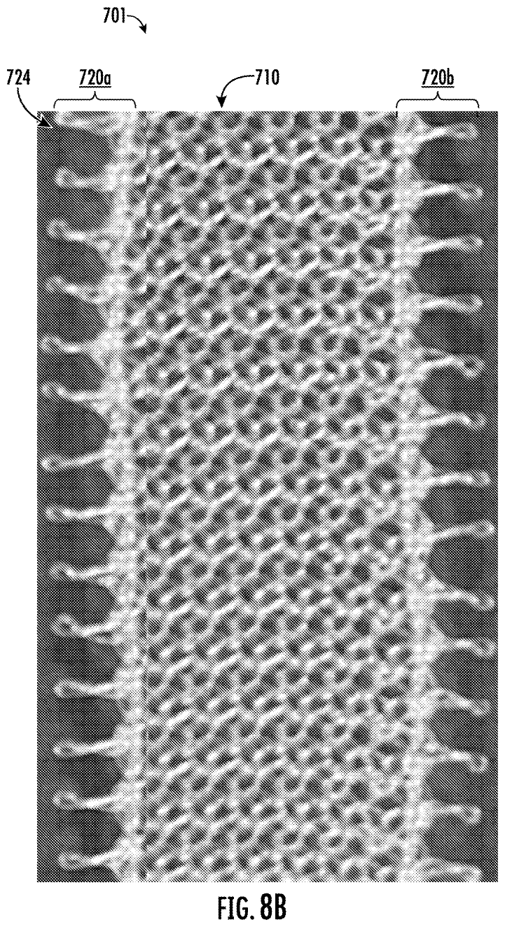

When each section 701 is ready to be separated from the mesh, the extensions 741 may be cut, as close to the anchor 740, or to the reinforced edge 720a 720b as desired, resulting in the section mesh 701 illustrated in FIG. 8A, for example. In some embodiments, the extensions 741 may be heat cut with a laser or similar, while in other embodiments the extensions 741 may be cut with a knife or shears. In some embodiments, the section mesh 701 may be intentionally unraveled to separate and leave exposed reinforced sections (e.g., 325 FIG. 3B) or barbed sections (e.g., 324 FIG. 3B). In this regard, each individual section 701 formed from the mesh may comprise reinforced edges 720a, 720b, defining a fray resistant mesh.

Returning to FIG. 7B, in some embodiments, to reduce the friction from the extensions 741, the extensions may be cut close to the reinforced edges 720a, 720b.

In another example embodiment, such as illustrated in FIG. 7C, the first section 701 and the second section 702 of the mesh 700″ may be connected to an intermediary mesh 742. In some embodiments, the intermediary mesh 742 may be similar to the anchor (e.g., 740 FIG. 7B), however, in some embodiments, it may define different extensions. In this regard, the intermediary mesh 742 may connect to the nibbed loops 724 of the reinforced edges 720a, 720b. Thus, when the intermediary mesh 742 is removed from the nibbed loops 724, the resulting mesh section 701 is available for use.

In some embodiments, such as illustrated in FIG. 8B, the barbed loops 724 remain intact when the intermediary mesh 742 is removed. In some embodiments, the barbed loops 724 may be partially severed when the intermediary mesh 742 is removed, however, due to the looping, any fragment of the loop created in the cut, will remain with the intermediary mesh 742. Thus, additional washing and/or agitation may be unnecessary to remove fragments, as the fragments may remain with the intermediary mesh 742 rather than the mesh section 701. Notably, as indicated herein, in some embodiments, the mesh may be formed with the barbed loops 724 intact and there may be no tacking yarn or other feature that is intended to be cut or otherwise removed.

FIGS. 7D-E illustrate additional configurations to connect discrete sections of the mesh 700″′, 700″″. FIG. 7D illustrates an example mesh 700″′ with a fourth yarn 7377 disposed between the second reinforcing edge 720b of the first section 701 and the first reinforcing edge 720a of the second section 702. In some embodiments, the fourth yarn 737 may be utilized in a chain stitch illustrated in FIG. 7D, or a looping stitch, such as illustrated in the mesh 700″″ in FIG. 7E. In some embodiments, the fourth yarn 737 may be the same material as the individual yarn (e.g., 115 FIG. 1), while in other embodiments the fourth yarn 737 may be a different material.

Although the methods described with reference to FIGS. 7A-8B are described in regards to separating sections of the mesh, it should be understood that each method and knit formation may be used to form one or more voids within a single section of mesh without the need to cut the mesh (e.g., FIG. 3C).

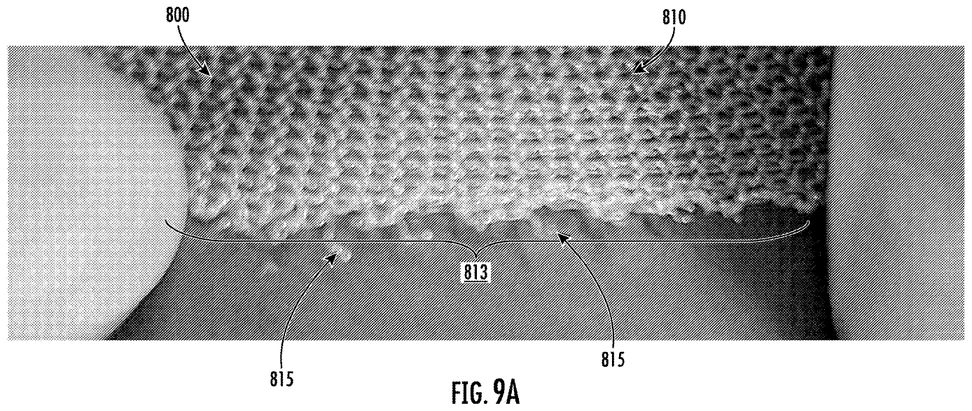

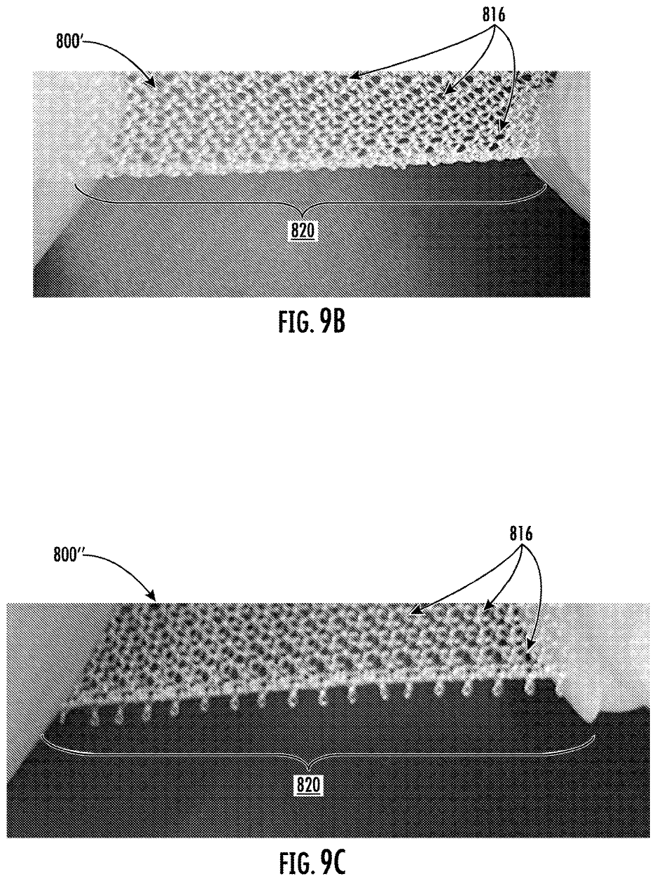

As described herein, the mesh formation defining a reinforced edge provides many benefits over other edge configurations. For example, the reinforced edge may provide the mesh with stretchability, while maintaining the shape and knit of the mesh. FIG. 9A illustrates a mesh 800 where the body 810 was cut at 813 (e.g., there was no reinforced edge). As illustrated, in the edge 813 where the cut was made, the individual yarns 815 are exposed, and there is no feature (e.g., reinforced edge) to keep the individual yarn 815 from unraveling as the mesh 800 is stretched. Although a heat cut may be used to partially melt the individual yarns 815, the melting creates rigidity and stiffness along the edge 813, which prevents stretching and setting into final forms. Further the heat cut edge 813 may be rough and abrasive and increase friction and abrasions to tissues and or objects positioned about the edge 813.

In contrast, as illustrated in FIGS. 9B-C, the mesh 800′, 800″ formed with the reinforced edge 820 is able to stretch into an extended position. As depicted in each of the figures, the reinforced edge 820 is able to extend, while maintaining the knit pattern, thereby yielding a softer, less abrasive edge, as pores 816 within the reinforced edge 820 are able to expand and contract with the pores 816 within the body 810 of the mesh 800′, 800″. Additionally, when the mesh 800′, 800″ is in the extended position, the mesh 800′, 800″ maintains its width, e.g., the spacing of the plurality of warp knit yarns within the body portion of the mesh. In this regard, the mesh will not narrow or neck when tension is applied in the machine direction.

In some embodiments, the warp knit mesh may be formed with sections separated by a void or a gap. In some embodiments, one or more tacking yarns may be used to connect and hold together otherwise separate sections. Such tacking yarns help in maintain desired structure and positioning during and after manufacture. In some embodiments, the one or more tacking yarns may be removable, such as via cutting and/or pulling thereon. In some embodiments, only a portion of a tacking yarn extending along a knit structure may be removed, with the remainder being left in the knit structure.

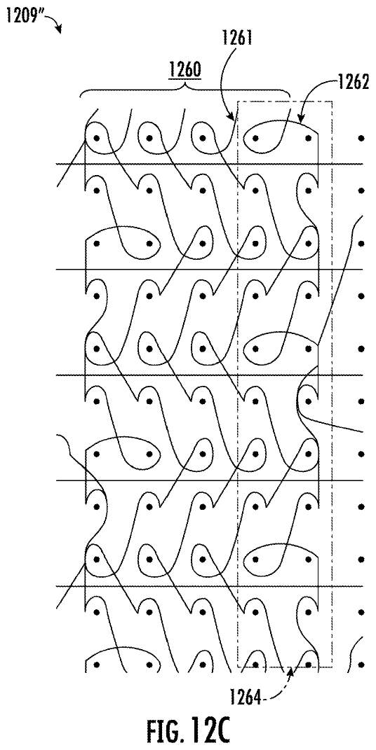

In some embodiments, each of the separate sections may extend from one or more overall edges, such as part of a band. In order to maintain the parallel position of each section in relation to one another, the edges of each section may be formed to maintain the length and structure of the section. FIGS. 12A-C illustrate example stitch notations 1209, 1209′, 1209″ forming different stitches for a section edge of the warp knit mesh.

In the illustrated embodiments, a plurality of first warp knit yarns 1260, including an end warp knit yarn 1261 positioned at an end of the plurality of first warp knit yarns 1260 may be positioned across a plurality of needles. Each of the plurality of first warp knit yarns 1260 may transition between at least two adjacent wales between two courses. In some embodiments, the plurality of first warp knit yarns 1260 may be formed in a first knit pattern. In some embodiments, in the first knit pattern, the end warp knit yarn 1261 may leave at least one needle point within the second wale uncovered in the transition between the first course and the second course. Specifically, the end warp knit yarn 1261 may transition between a first wale and a second wale, between each course, and, in every other course, at least one needle within the second wale may be uncovered due to the transition of the end warp knit yarn 1261.

A section edge 1264 of the first section 1201 may be formed by at least one second warp knit yarn 1262. The at least one second warp knit yarn 1262 may extend adjacent to the end warp knit yarn 1261. The at least one second warp knit yarn 1262 may be formed into a second knit pattern, which is different than the first knit pattern. As discussed above, in some embodiments the first knit pattern may leave at least one needle point within the second wale uncovered in the transition between the first course and the second course. In some embodiments, the at least one second warp knit yarn may loop around (or otherwise cover) the at least one needle point within the second wale, which was left uncovered by the first knit pattern. Specifically, in some embodiments the second knit pattern may comprise single loops and underlaps along the length of the edge, while in other embodiments the second knit pattern comprises at least one double loop or underlap along the length of the edge of the section 1201. In this regard, the second knit pattern may cover any of the needle points which are left uncovered by the plurality of first warp knit yarns within the first section 1201 and may not extend past a certain wale (e.g., the last illustrated wale) to form a smooth edge 1264. The at least one second warp knit yarn 1262 thereby forms a smooth edge of the first section 1201.

FIG. 12A illustrates a stitch notation 1209 for a first section edge configuration. In the first section edge configuration the plurality of first warp knit yarns 1260 may each transition between two adjacent wales between each course. The end warp knit yarn 1261, which may transition between a first wale and a second wale, leaves a needle open on the second wale between every other course. Thus, the at least one second warp knit yarn 1262 may be knit in a second pattern, to cover the needle left open by the end warp knit yarn 1261. In this embodiment, the at least one second warp knit yarn covers a single wale between each course, to form the section edge 1264.

A second section edge configuration is illustrated in FIG. 12B. In some embodiments, the at least one second warp knit yarn 1262 may cover more than one wale. In this regard, illustrated in stitch notation 1209′, the at least one second warp knit yarn 1262 may cover the second wale and a further positioned third wale. In this regard, the at least one second warp knit yarn 1262 may cover the second wale and the further positioned third wale in the courses where the end warp knit yarn 1261 is on the first wale, and the at least one second warp knit yarn 1262 may cover only the further positioned third wale in the courses where the end warp knit yarn 1261 is on the second wale.

FIG. 12C illustrates another stitch notation 1209″ for a third section edge configuration. In some embodiments, each of the plurality of first warp knit yarn may extend across three wales between three courses. In this regard, the end yarn 1261 may transition between a first wale, a second wale, and a further positioned third wale, across three courses, and then transition back from the further positioned third wale to the first wale, along another three courses. In such embodiments the at least one second warp knit yarn 1262 may be positioned about the second wale and the further positioned third wale while not extending to a subsequent further positioned fourth wale. To create the section edge 1264, the at least one second warp knit yarn may transition from covering both the second wale and the further positioned third wale, (e.g., when the end warp knit yarn 1261 covers the first wale), to covering only the further positioned third wale (e.g., when the end warp knit yarn 1261 covers the second wale) to not covering a needle in the further positioned third wale (e.g., when the end warp knit yarn 1261 covers the further positioned third wale).

In some embodiments, an extension knit pattern may be formed from at least one third warp knit yarn. With reference to FIGS. 8A-B, in some embodiments, the extension knit pattern may comprise extensions 710, while in other embodiments, the extension knit pattern may comprise nibs or barbs 724.

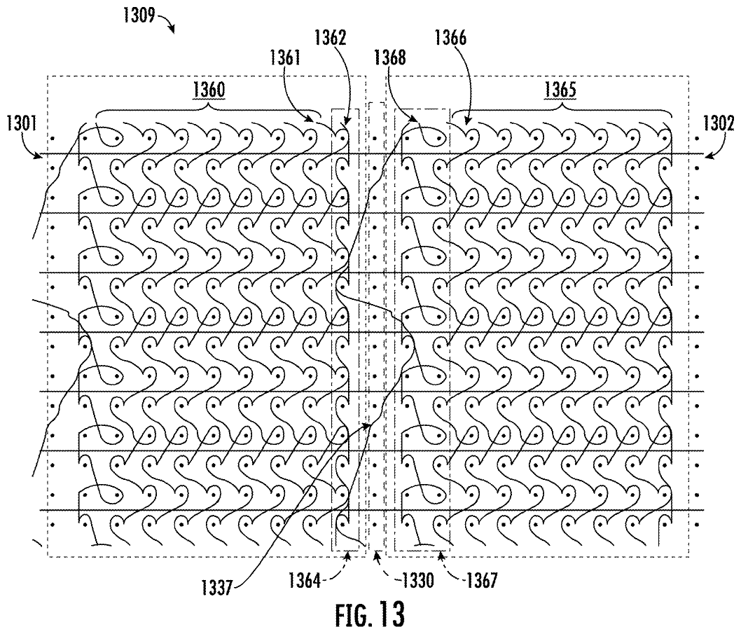

A warp knit mesh may be formed from multiple sections joined together. FIG. 13 illustrates a stitch notation 1309 to form a warp knit mesh comprising more than one section. As illustrated in FIG. 13, a first section 1301 of warp knit yarn is formed from a plurality of first warp knit yarns 1360, including an end warp knit yarn 1361 and at least one second warp knit yarn 1362 positioned adjacent the end warp knit yarn 1361. Similarly, a second section 1302 is formed from a plurality of second warp knit yarns 1365, including an end warp knit yarn 1366 of the plurality of second warp knit yarns 1365, and at least one third warp knit yarn 1368 extending adjacent to the end warp knit yarn 1366 of the plurality of second warp knit yarns 1365.

Each of the first section 1301 and the second section 1302 may define a section edge with a configuration of, for example, one of the edge configurations described with reference to FIGS. 12A-C (although other edge configurations are contemplated herein). In this regard, the first section 1301 may define a first section edge 1364 and the second section 1302 may define a second section edge 1367. In some embodiments, the first section edge 1364 and the second section edge 1367 may have the same configuration, while in other embodiments, the first section edge 1364 and the second section edge 1367 may define different edge configurations. Notably, however, in various embodiments, both section edges are smooth without cut yarns.

Thus, in some embodiments, the plurality of second warp knit yarns 1365 may be formed in a third knit pattern, and the end warp knit yarn 1368 of the plurality of second warp knit yarns 1365 may be formed into a fourth knit pattern. In some embodiments, the first knit pattern and the third knit pattern may be the same, while in other embodiments the first knit pattern and the third knit pattern may be different. Similarly, in some embodiments the second knit pattern and the fourth knit pattern may be the same, while in other embodiments the second knit pattern and the fourth knit pattern may be different.

In some embodiments, the end warp knit yarn 1368 of the plurality of second warp knit yarns 1365 may be formed between a third wale and a fourth wale. In this regard, the third wale, may be similar to the first wale, while the fourth wale may be similar to the second wale as described with reference to FIGS. 12A-C.

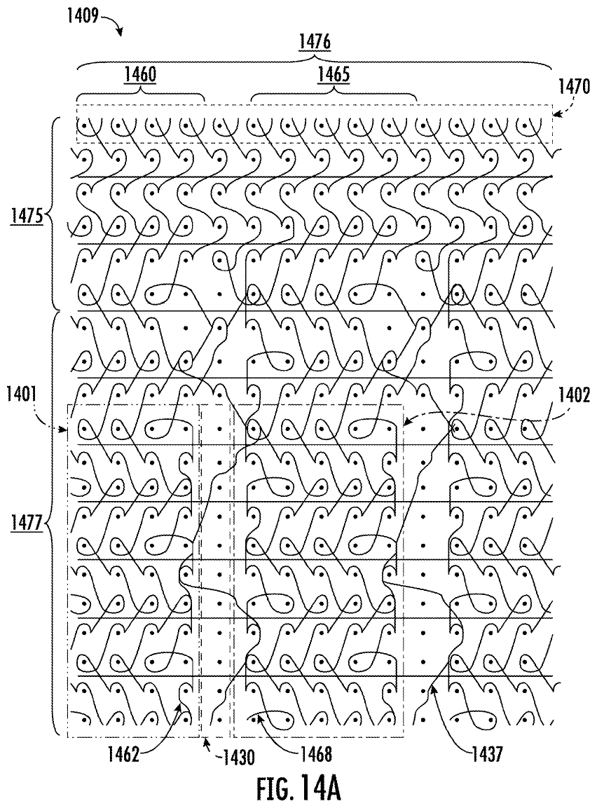

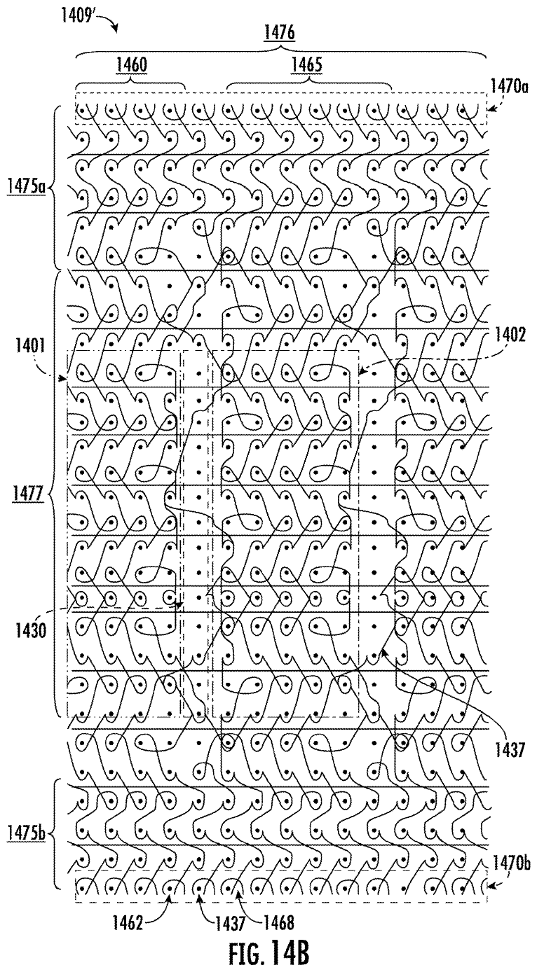

In some embodiments, the first section 1301 and the second section 1302 may be connected at a band (e.g., 1475 FIG. 14A) defining an overall edge (e.g., 1470 FIG. 14A).

Returning to reference FIG. 13, the band may extend for a number of courses before transitioning in to the first section 1301 and the second section 1302. In some embodiments, the first section 1301 and the second section 1302 may be separated by a void 1330. In some embodiments, the void 1330 may be formed in the formation of the warp knit yarn (e.g., as illustrated in the stitch notation 1309), while in other embodiments the void 1330 may be formed by removing a portion of the mesh (e.g., cutting) between the first section edge 1364 and the second section edge 1367.

In some embodiments, a tacking yarn 1337 may be positioned between the first section edge 1364 and the second section edge 1367. The tacking yarn 1337 may removably connect the first section 1301 and the second section 1302. In this regard, the tacking yarn 1337 may be removed after formation of the warp knit mesh. Further, the tacking yarn 1337 may be removed, such that no filament, or residual yarn is left within the warp knit mesh, after the tacking yarn 1337 is cut and/or removed.

FIG. 14A illustrates a stitch notation 1409 for forming a warp knit mesh. The warp knit mesh may comprise a band section 1475 and a strip section 1477. In some embodiments, the strip section 1475 may comprise at least a first strip 1401 and a second strip 1402 extending from the band 1475. As used herein it should be understood that the first strip 1401 and the second strip 1402 may be akin to the first section 1301 and the second section 1302 described with reference to FIG. 13.

In some embodiments, the band 1475 may comprise a plurality of warp knit yarns 1476 extending in a first knit pattern. In some embodiments, the plurality of warp knit yarns may include a first set of warp knit yarns 1460 and a second set of warp knit yarns 1465, each of the sets of yarns comprising different yarns. In some embodiments, the plurality of warp knit yarns 1476 may further include one or more tacking yarns 1737. In some embodiments, the number of tacking yarns is less than the number of strips formed within the strip section 1477.

In some embodiments, the warp knit mesh may transition between the band section 1475 and the strip section 1477 over a single course, while in other embodiments multiple courses may be needed to transition the warp knit mesh between the band section 1475 and the strip section 1477. In the transition between the band section 1475 and the strip section 1477 the one or more tacking yarns 1437, the first set of warp knit yarns 1460, and the second set of warp knit yarns 1465 may change knit patterns.

To explain, the first strip 1401 may be formed of the first set of warp knit yarns 1460 and the second strip 1402 may be formed from the second set of warp knit yarns 1465. Each of the first strip 1401 and the second strip 1402 may be formed in a second knit pattern (although, in some embodiments, the second knit patterns could be different between strips), wherein the second knit pattern is different that the first knit pattern. Thus, when the first set of warp knit yarns 1460, and the second set of warp knit yarns 1465 are within the band section 1475 (e.g., in the plurality of warp knit yarns), the first set of warp knit yarns 1460 and the second set of warp knit yarns 1465 are knit in the first knit pattern. However, after the determined number of courses necessary to form the band section 1475, each of the first set of warp knit yarns 1460 and the second set of warp knit yarns 1465 transition from the first knit pattern to the second knit pattern.

Returning to the strip configuration, in some embodiments, the second knit pattern may comprise a body knit pattern and an edge knit pattern, each of the knit patterns being different. In this regard, the body knit pattern may correspond to the first knit pattern and the edge knit pattern may correspond to the second knit pattern described with reference to FIGS. 12A-C, and FIG. 13.

In the plurality of warp knit yarns, one of the one or more tacking yarns 1437 is positioned between the first set of warp knit yarn 1460 and the second set of warp knit yarn 1465. However, the one or more tacking yarns 1437 is not included in either the first strip 1401 or the second strip 1402. In this regard, rather than being integrated into either the first strip 1401 or the second strip 1402 along every course, the one or more tacking yarns 1437 may loosely transition between the first strip 1401 and the second strip 1402 over multiple courses, forming a tacking section therebetween. In this regard, the one or more tacking yarns 1437 may be configured to aid in holding the first strip 1401 and the second strip 1402 parallel to one another, but may not be integral to, and may be easily removable from each of the first strip 1401 and the second strip 1402.

After the one or more tacking yarns 1437 are removed from the tacking section of the mesh, a gap 1430 is formed between the first strip 1401 and the second strip 1402. The gap 1430 may correspond to at least one pick position (e.g., one wale) between an edge 1462 of the first strip 1401 and an edge 1468 of the second strip 1402. In some embodiments, the gap 1430 may be fully formed upon removal of the one or more tacking yarns 1437. In this regard, after formation of the warp knit mesh, the one or more tacking yarns 1437 may be removed from adjacent strips, for example, the first strip 1401 and the second strip 1402. Thus, the one or more tacking yarn 1437 may be cut at the transition between the band section 1475 and the strip section 1477 and removed such that no cut yarn remains between adjacent strips, while remaining knit in the first knit pattern within the band section 1475.

FIG. 14B illustrates an example stitch notation 1409′ for forming a warp knit mesh similar to that of FIG. 14A, however, the stitch notation 1409′ illustrates use of a top band 1475a and a bottom band 1475b. Notably, the strip section 1477 can thus transition between and be held between the two bands (e.g., the top band 1475a and the bottom band 1475b).

While various example embodiments illustrate straight edges, in some embodiments, more complex shapes can be formed using reinforced edges. In this regard, different knitting techniques can be used to formed angled or tapered edges according to various example embodiments.

Various embodiments of the present invention provide mesh structures that include reinforced edges to aid in preventing or limited fraying and unraveling. Notably, various embodiments of the present invention may utilize different characteristics of the yarns and/or overall mesh, such as may further aid in providing a desirable mesh structure. For example, reinforcement edge strength and/or nib/barb strength may be designed into the reinforced edges. As another example, the mesh may be designed with certain levels of tension or shape stability. In some embodiments, the mesh may be designed to be formable with minimal excess filament fragment formation, generating only a certain number of cut yarns per inch of cut edge, for example.

### Example Flowchart(s) and Operations

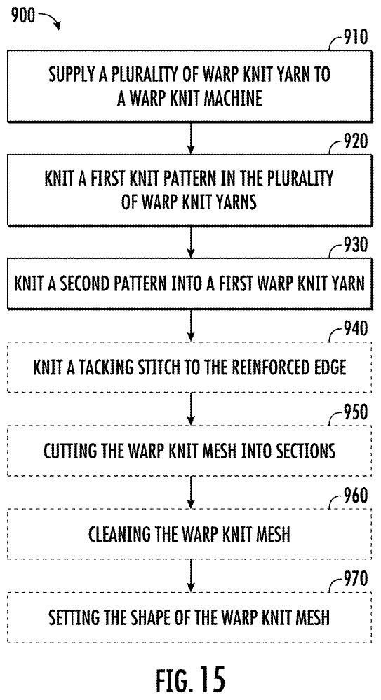

Some embodiments of the present invention provide methods, and apparatus related to forming various mesh structures according to various embodiments described herein. Various examples of the operations performed in accordance with embodiments of the present invention are herein provided with reference to FIG. 15.

FIG. 15 illustrates a flowchart according to an example method 900 of forming a mesh. At operation 910, a plurality of warp knit yarns are supplied to a warp knit machine. In some embodiments, the plurality of warp knit yarn may include a first warp knit yarn. At operation 920, a first knit pattern is knit into the plurality of warp knit yarns leading to the first warp knit yarn. At operation 930, a second knit pattern is knit into the first warp knit yarn to form a reinforced edge.

Optionally at operation 940, a tacking stitch may be applied to the reinforced edge to temporarily connect the mesh section with the reinforced edge to another section of the mesh. Optionally at operation 950, the warp knit mesh may be cut into sections, such as along the tacking stitch. In some embodiments, the warp knit mesh may be cut with a sharp device, for example a knife, or shears. While in other embodiments the warp knit mesh may be cut with a heating device, for example, a laser.

Optionally at operation 960, the warp knit mesh may be cleaned. Optionally at operation 970, the mesh may be shaped. In some embodiments, cleaning the mesh may include submerging the mesh into water and shaking the mesh a predetermined number of times. In some embodiments, cleaning the mesh may allow any excess cut yarns to be removed from the mesh. In some embodiments, to set and shape the mesh, the mesh may be stretched such that the pores within the mesh exhibit the desired size. Thereafter, the mesh may be exposed to a heating treatment or a thermal treatment.

Although described herein as individual operations, it should be understood that in some embodiments multiple operations may be incorporated into a single operation, and/or performed simultaneously (e.g., operations 920-940 may be performed simultaneously and/or in different orders/sequences). In this regard, in some embodiments, the operations may be performed in any order.

### Conclusion

Many modification and other embodiments of the inventions set forth herein will come to mind to one skilled in the art to which these inventions pertain having the benefit of the teaching presented in the foregoing description and the associated drawings. Therefore, it is to be understood that the embodiments of the invention are not to be limited to the specific embodiments disclosed and that the modification and other embodiments are intended to be included within the scope of the invention. Moreover, although the foregoing descriptions and the associated drawings describe example embodiments in the context of certain example combinations of elements and/or functions may be provided by alternative embodiments without departing from the scope of invention. In this regard, for example, different combinations of elements and/or functions than those explicitly described above are also contemplated within the scope of the invention. Although specific terms are employed herein, they are used in a generic and descriptive sense only and not for purposes of limitation.

1-42. (canceled)

43. A warp knit mesh comprising:
a first section, the first section comprising:
a plurality of first warp knit yarns formed into a first knit pattern, wherein at least a portion of the first knit pattern is designed such that an end warp knit yarn of the plurality of first warp knit yarns crosses from a first wale to a second wale when transitioning between a first course and an adjacent second course; and
at least one second warp knit yarn arranged adjacently to the end warp knit yarn of the plurality of first warp knit yarns and formed into a second knit pattern, wherein the first knit pattern is different than the second knit pattern, wherein at least a portion of the second knit pattern is designed such that the at least one second warp knit yarn forms a section edge for the first section for at least the first course and the second course by either (i) stopping at the second wale without extending to any needle points within the first course or the second course for a further adjacent third wale, or (ii) forming the section edge for the first section for at least the first course and the second course along a further positioned third wale by stopping at the further positioned third wale without extending to any needle points within the first course or the second course for a subsequent further positioned fourth wale, wherein the subsequent further positioned fourth wale is adjacent to the further positioned third wale.

44. The warp knit mesh of claim 43, wherein the section edge is smooth without cut yarns.

45. A warp knit mesh comprising:
a band comprising a plurality of warp knit yarns defining at least a first knit pattern, wherein the plurality of warp knit yarns comprises at least a first set of warp knit yarns and a second set of warp knit yarns, wherein the first set of warp knit yarns are different than the second set of warp knit yarns; and
a plurality of strips extending from the band, wherein the plurality of strips includes at least a first strip and a second strip,
wherein the first strip is formed of the first set of warp knit yarns, wherein the first set of warp knit yarns define a second knit pattern within the first strip that is different than the first knit pattern;
wherein the second strip extends from the band in a parallel fashion to the first strip, the second strip formed of the second set of warp knit yarns, wherein the second set of warp knit yarns define the second knit pattern,
wherein the first strip and the second strip are separated by a gap therebetween, wherein the gap corresponds to at least one pick position between an edge of the first strip and an edge of the second strip.

46. The warp knit mesh of claim 45, wherein the plurality of warp knit yarns forming the band further comprises one or more tacking warp knit yarns, wherein the one or more tacking warp knit yarns, the first set of warp knit yarns, and the second set of warp knit yarns together form the first knit pattern, and wherein the one or more tacking warp knit yarns extend within the gap and are removable.

47. The warp knit mesh of claim 46, wherein the one or more tacking warp knit yarns are reengaged into the first knit pattern within the band.

48. The warp knit mesh of claim 45, wherein the first set of warp knit yarns transition between the first knit pattern and the second knit pattern between a first course and a second course.

49. The warp knit mesh of claim 45, wherein the mesh is designed such that there are no cut yarns between the first strip and the second strip.

50. The warp knit yarn of claim 45, wherein the second knit pattern comprises an edge knit pattern and a body knit pattern, wherein the edge knit pattern and the body knit pattern are different.

51. The warp knit yarn of claim 50, wherein the first strip comprises a first edge, wherein the second strip comprises a second edge, wherein the first edge and the second edge comprise the edge knit pattern, wherein the edge knit pattern is continuous along a length of the first strip or the second strip, and wherein the edge knit pattern is smooth such that there are no cut yarns.

52. The warp knit yarn of claim 51, wherein the edge knit pattern comprises single loops and underlaps along the length of the first strip or the second strip.

53. The warp knit yarn of claim 51, wherein the edge knit pattern comprises at least one double loop, overlap, or underlap along the length of the first strip or the second strip.

54. A method of forming a knit structure, the method comprising:
knitting a plurality of warp knit yarns in a first knit pattern to form a band within the knit structure, wherein each of the warp knit yarns within the plurality of warp knit yarns is used in the first knit pattern; and
transitioning the plurality of warp knit yarns from the first knit pattern to a second knit pattern forming at least a first strip, a second strip, and a tacking yarn positioned therebetween, wherein, in the second knit pattern, at least one warp knit yarn of the plurality of warp knit yarns becomes the tacking yarn.

55. The method of claim 54, wherein the first strip is formed of a first set of the plurality of warp knit yarns, and the second strip is formed of a second set of the plurality of warp knit yarns.

56. The method of claim 54, wherein the second knit pattern comprises an edge knit pattern and a body knit pattern, wherein the edge knit pattern and the body knit pattern are different, wherein the edge knit pattern is smooth so as to define no cut yarns.

57. The method of claim 54, wherein the first strip comprises a first edge, wherein the second strip comprises a second edge, wherein the first edge and the second edge comprise the edge knit pattern, wherein the edge knit pattern is continuous along a length of the first strip or the second strip.

58. The method of claim 54 further comprising removing the tacking yarn between the first strip and the second strip, wherein the tacking yarn remains knit within the first knit pattern.

59. The method of claim 54, wherein the knit structure is designed such that no cut yarns are formed between the first strip and the second strip.

60. The method of claim 54, further comprising cleaning the knit structure.

61. The method of claim 54, further comprising setting the knit structure into a final shape.

62. The method of claim 61, wherein setting the knit structure comprises a thermal process or a heating process.

63. A warp knit mesh comprising:
a first section, the first section comprising:
a plurality of warp knit yarns extending in side-by side relation, wherein the plurality of warp knit yarns comprises at least a first set of warp knit yarns, a second set of warp knit yarns, and one or more tacking warp knit yarns that together form a first knit pattern within the first section; and
a second section, the second section comprising:
a first strip defined by the first set of the plurality of warp knit yarns, wherein the first set of the plurality of warp knit yarns extend in side-by-side relation in a second knit pattern that is different than the first knit pattern,
a second strip defined by the second set of the plurality of warp knit yarns, wherein the second set of the plurality of warp knit yarns extend in side-by side relation in the second knit pattern; and
a tacking section defined by the one or more tacking warp knit yarn formed into a third knit pattern that is different than the first knit pattern and the second knit pattern,
wherein the first set of the plurality of warp knit yarns, the second set of the plurality of warp knit yarns, and the one or more tacking warp knit yarns transition together from the first knit pattern to the respective second knit patterns and third knit pattern across one or more courses.

64. The warp knit mesh of claim 63, wherein the one or more tacking warp knit yarn is removable in relation to the first strip and the second strip so as to form a gap between the first strip and the second strip.
